SEQUENCE LISTING (6 sequences)

SEQ ID NO: 1 - DNA, 24 bp, Artificial Sequence
  synthetic DNA
attattattatgtggttgctgtgt

SEQ ID NO: 2 - DNA, 22 bp, Artificial Sequence
  synthetic DNA
attattaagcgtgtactgaact

SEQ ID NO: 3 - DNA, 27 bp, Artificial Sequence
  synthetic DNA
tacaccaacgacacaaatgttattagg

SEQ ID NO: 4 - DNA, 27 bp, Artificial Sequence
  synthetic DNA
tcgcacatgacttgaaatgttattagg

SEQ ID NO: 5 - DNA, 22 bp, Artificial Sequence
  synthetic DNA
ttacaataatccattattatta

SEQ ID NO: 6 - DNA, 22 bp, Artificial Sequence
  synthetic DNA
ttacaataatccattattatta

(19) United States
(12) Patent Application Publication
(10) Pub. No.: US 2011/0195526 A1
(43) Pub. Date: Aug. 11, 2011

(54) Thermo-probes, methods of making thermo-probes, and methods of detection using thermo-probes

(75) Inventors: Ming Su, Oviedo, FL (US); Liyuan Ma, Orlando, FL (US); Yan Hong, Orlando, FL (US)

(73) Assignee: University of Central Florida Research Foundation, Orlando, FL (US)

(21) Appl. No.: 12/931,864

(22) Filed: Feb. 11, 2011

Related U.S. Application Data
(60) Provisional application No. 61/303,396, filed on Feb. 11, 2010.

(51) Int. Cl. G01N 33/543; G01N 30/00; B82Y 15/00

(52) U.S. Cl. 436/526; 436/518; 422/69; 977/773

(57) ABSTRACT

Embodiments of the present disclosure provide for thermo-probes (also referred to herein as “probes”), particles including a plurality of probes, methods of making thermo-probes or particles, thermo-probe complexes, method of making thermo-probe complexes, methods of detecting a target, methods of detecting multiple targets, assays using the thermo-probes, immunoassays using the thermo-probes, methods of using thermo-probes or particles, and the like.

### CROSS-REFERENCE TO RELATED APPLICATIONS

This application claims priority to U.S. provisional application entitled, “THERMO-PROBES, METHODS OF MAKING THERMO-PROBES, AND METHODS OF DETECTION USING THERMO-PROBES,” having Ser. No. 61/303,396, filed on Feb. 11, 2010, which is entirely incorporated herein by reference.

### STATEMENT REGARDING FEDERALLY SPONSORED RESEARCH OR DEVELOPMENT

This invention was made with government support under Grant No.: 07KN11, 0828466, W81XWH-10-1-0961 awarded by the Florida Department of Health, National Science Foundation, and the Department of Defense, respectively. The government has certain rights in the invention.

### BACKGROUND

A major challenge of biomarker-based early detection of cancer is that some biomarkers (nucleic acids or proteins) exist at low concentration and are not effective in reflecting cancers (Nature Rev. Cancer 5, 161 (2005)). Multiplexed highly sensitive detection of multiple biomarkers has been proposed to enhance the accuracy (Curr. Opin. Mol. Ther. 9, 563 (2007)). Nanoparticles with unique magnetic, electric, optical, and electrochemical signatures have been used to detect biomarkers with high sensitivity by converting biological events into physical signals that can be amplified (Chem. Rev. 105, 1547 (2005); Science 301, 1884 (2003); ACS Nano 3, 585 (2009); Science 277, 1078 (1997)), but the detection multiplicity is limited due to narrow detection range and wide peak (Appl. Phys. Lett. 82, 3562 (2003)). Only one or few types of biomarkers can be detected each time, and screening one sample for multiple biomarkers takes a long time. Magnetic nanoparticles lack particle-specific property for multiplicity (Angew. Chem. Int. Ed. 40, 3204 (2001)). Electric measurement cannot distinguish two types of metallic nanoparticles (Science 295, 1503 (2002)). Although few types of nanoparticles can be detected by electrochemical method (Anal. Chem. 73, 5576 (2001)), the method spans several volts, and the peaks are too broad to offer large multiplicity. The absorbance peaks of optically-active nanoparticles in UV-visible region (400 to 900 nm) are broad with peak width at half height of 150 nm (Nature Biotechnol. 22, 47 (2004); J. Am. Chem. Soc. 128, 2526 (2006); Nanomedicine 3, 543 (2008); Nature Biotechnol. 19, 631 (2001)), which limits the detection capacity. In case of surface enhanced Raman spectroscopy where sharp peaks exist over large range, the available Raman active dyes are limited (Science 297, 1536 (2002)), the ratio of Raman “hot” nanoparticles is low, and quantitative signals are hard to obtain. Thus, there is a need to find alternatives.

### SUMMARY

Embodiments of the present disclosure provide for thermo-probes (also referred to herein as “probes”), particles including a plurality of probes, methods of making thermo-probes or particles, thermo-probe complexes, method of making thermo-probe complexes, methods of detecting a target, methods of detecting multiple targets, assays using the thermo-probes, immunoassays using the thermo-probes, methods of using thermo-probes or particles, and the like.

An embodiment of the probe, among others, includes: a nanoparticle having an outer structure and an inner area, wherein the outer structure encapsulates the inner area, wherein a phase change material is within the inner area, wherein the melting point of the outer structure is greater than the melting point of the phase change material, wherein a first targeting moiety is attached to the surface of the outer structure.

An embodiment of the probe complex, among others, includes: a probe, a capture moiety, and a target, wherein the probe is a nanoparticle having an outer structure and an inner area, wherein the outer structure encapsulates the inner area, wherein a phase change material is within the inner area, wherein the melting point of the outer structure is greater than the melting point of the phase change material, wherein a targeting moiety is attached to the surface of the outer structure, wherein a second targeting moiety is attached to the capture moiety, wherein the targeting moiety has an affinity for a first portion of the target, wherein the second targeting moiety has an affinity for a second portion of the target, wherein the first portion of the target and the second portion of the target are not the same.

An embodiment of the particle, among others, includes: a plurality of nanoparticles enclosed by a particle shell structure, wherein each nanoparticle has an outer structure and an inner area, wherein the outer structure encapsulates the inner area, wherein a phase change material is within the inner area, wherein the melting point of the outer structure is greater than the melting point of the phase change material.

An embodiment of the method of detecting a target, among others, includes: mixing a probe, a capture moiety, and a target, wherein the probe is a nanoparticle having an outer structure and an inner area, wherein the outer structure encapsulates the inner area, wherein a phase change material is within the inner area, wherein the melting point of the outer structure is greater than the melting point of the phase change material, wherein a first targeting moiety is attached to the surface of the outer structure, wherein a second targeting moiety is attached to the capture moiety, wherein the first targeting moiety has an affinity for a first portion of the target, wherein the second targeting moiety has an affinity for a second portion of the target, wherein the first portion of the target and the second portion of the target are not the same; forming a probe complex including the probe, the second targeting moiety, and the target, wherein the first targeting moiety is attached to the first portion of the target, wherein the second targeting moiety is attached to the second portion of the target; separating the probe complex from the other components of the mixture; heating the probe complex; and determining the melting point of the phase change material, wherein the melting point is used to identify the phase change material, wherein identifying the phase change material is used to determine the target.

An embodiment of the method of detecting a target, among others, includes: mixing a first probe, a second probe, a first capture moiety, a first target, and a second target, wherein the first probe is a nanoparticle having an outer structure and an inner area, wherein the outer structure encapsulates the inner area, wherein a first phase change material is within the inner area, wherein the melting point of the outer structure is greater than the melting point of the first phase change material, wherein a first targeting moiety is attached to the surface of the outer structure, wherein the second probe is a nanoparticle having an outer structure and an inner area, wherein the outer structure encapsulates the inner area, wherein a second phase change material is within the inner area, wherein the melting point of the outer structure is greater than the melting point of the second phase change material, wherein the melting point of the first phase change material is different than the melting point of the second phase change material, wherein a second targeting moiety is attached to the surface of the outer structure, wherein a third targeting moiety is attached to the first capture moiety, wherein the first targeting moiety has an affinity for a first portion of the first target, wherein the third targeting moiety has an affinity for a second portion of the first target, wherein the first portion of the first target and the second portion of the first target are not the same, wherein the second targeting moiety has an affinity for a first portion of the second target, wherein the third targeting moiety has an affinity for a second portion of the second target, wherein the first portion of the second target and the second portion of the second target are not the same; forming a first probe complex including the first probe, the third targeting moiety, and the first target, wherein first targeting moiety is attached to the first portion of the first target, wherein the third targeting moiety is attached to the second portion of the first target; forming a second probe complex including the second probe, the third targeting moiety, and the second target, wherein the second targeting moiety is attached to the first portion of the second target, wherein the third targeting moiety is attached to the second portion of the second target; separating the first probe complex and the second probe complex from the other components of the mixture; heating the first probe complex and the second probe complex; determining a first melting point of the first phase change material, wherein the first melting point is used to identify the first phase change material, wherein identifying the first phase change material is used to determine the first target; and determining a second melting point of the second phase change material, wherein the second melting point is used to identify the second phase change material, wherein identifying the second phase change material is used to determine the second target.

An embodiment of the method of detecting a target, among others, includes: mixing a first probe, a second probe, a first capture moiety, a second capture moiety, a first target, and a second target, wherein the first probe is a nanoparticle having an outer structure and an inner area, wherein the outer structure encapsulates the inner area, wherein a first phase change material is within the inner area, wherein the melting point of the outer structure is greater than the melting point of the first phase change material, wherein a first targeting moiety is attached to the surface of the outer structure, wherein the second probe is a nanoparticle having an outer structure and an inner area, wherein the outer structure encapsulates the inner area, wherein a second phase change material is within the inner area, wherein the melting paint of the outer structure is greater than the melting point of the second phase change material, wherein the melting point of the first phase change material is different than the melting point of the second phase change material, wherein a second targeting moiety is attached to the surface of the outer structure, wherein a third targeting moiety is attached to the first capture moiety, wherein a fourth targeting moiety is attached to the second capture moiety, wherein the first targeting moiety has an affinity for a first portion of the first target, wherein the third targeting moiety has an affinity for a second portion of the first target, wherein the first portion of the first target and the second portion of the first target are not the same, wherein the second targeting moiety has an affinity for a first portion of the second target, wherein the fourth targeting moiety has an affinity for a second portion of the second target, wherein the first portion of the second target and the second portion of the second target are not the same; forming a first probe complex including the first probe, the third targeting moiety, and the first target, wherein first targeting moiety is attached to the first portion of the first target, wherein the third targeting moiety is attached to the second portion of the first target; forming a second probe complex including the second probe, the fourth targeting moiety, and the second target, wherein the second targeting moiety is attached to the first portion of the second target, wherein the fourth targeting moiety is attached to the second portion of the second target; separating the first probe complex and the second probe complex from the other components of the mixture; heating the first probe complex and the second probe complex; determining a first melting point of the first phase change material, wherein the first melting point is used to identify the first phase change material, wherein identifying the first phase change material is used to determine the first target; and determining a second melting point of the second phase change material, wherein the second melting point is used to identify the second phase change material, wherein identifying the second phase change material is used to determine the second target.

An embodiment of the method of detecting a target, among others, includes: mixing a probe, a capture moiety, and a target, wherein the probe is a nanoparticle made of a phase change material, wherein a first targeting moiety is attached to the surface of the nanoparticle, wherein a second targeting moiety is attached to the capture moiety, wherein the first targeting moiety has an affinity for a first portion of the target, wherein the second targeting moiety has an affinity for a second portion of the target, wherein the first portion of the target and the second portion of the target are not the same; forming a probe complex including the probe, the second targeting moiety, and the target, wherein the first targeting moiety is attached to the first portion of the target, wherein the second targeting moiety is attached to the second portion of the target; separating the probe complex from the other components of the mixture; heating the probe complex; and determining the melting point of the phase change material, wherein the melting point is used to identify the phase change material, wherein identifying the phase change material is used to determine the target.

An embodiment of the method of detecting a target, among others, includes: mixing a first probe, a second probe, a first capture moiety, a first target, and a second target, wherein the first probe is a nanoparticle made of a first phase change material, wherein a first targeting moiety is attached to the surface of the nanoparticle, wherein the second probe is a nanoparticle made of a second phase change, wherein the melting point of the first phase change material is different than the melting point of the second phase change material, wherein a second targeting moiety is attached to the surface of the nanoparticle, wherein a third targeting moiety is attached to the first capture moiety, wherein the first targeting moiety has an affinity for a first portion of the first target, wherein the third targeting moiety has an affinity for a second portion of the first target, wherein the first portion of the first target and the second portion of the first target are not the same, wherein the second targeting moiety has an affinity for a first portion of the second target, wherein the third targeting moiety has an affinity for a second portion of the second target, wherein the first portion of the second target and the second portion of the second target are not the same; forming a first probe complex including the first probe, the third targeting moiety, and the first target, wherein first targeting moiety is attached to the first portion of the first target, wherein the third targeting moiety is attached to the second portion of the first target; forming a second probe complex including the second probe, the third targeting moiety, and the second target, wherein the second targeting moiety is attached to the first portion of the second target, wherein the third targeting moiety is attached to the second portion of the second target; separating the first probe complex and the second probe complex from the other components of the mixture; heating the first probe complex and the second probe complex; determining a first melting point of the first phase change material, wherein the first melting point is used to identify the first phase change material, wherein identifying the first phase change material is used to determine the first target; and determining a second melting point of the second phase change material, wherein the second melting point is used to identify the second phase change material, wherein identifying the second phase change material is used to determine the second target.

An embodiment of the method of detecting a target, among others, includes: mixing a first probe, a second probe, a first capture moiety, a second capture moiety, a first target, and a second target, wherein the first probe is a nanoparticle made of a first phase change material, wherein a first targeting moiety is attached to the surface of the nanoparticle, wherein the second probe is a nanoparticle made of a second phase change material, wherein the melting point of the first phase change material is different than the melting point of the second phase change material, wherein a second targeting moiety is attached to the surface of the nanoparticle, wherein a third targeting moiety is attached to the first capture moiety, wherein a fourth targeting moiety is attached to the second capture moiety, wherein the first targeting moiety has an affinity for a first portion of the first target, wherein the third targeting moiety has an affinity for a second portion of the first target, wherein the first portion of the first target and the second portion of the first target are not the same, wherein the second targeting moiety has an affinity for a first portion of the second target, wherein the fourth targeting moiety has an affinity for a second portion of the second target, wherein the first portion of the second target and the second portion of the second target are not the same; forming a first probe complex including the first probe, the third targeting moiety, and the first target, wherein first targeting moiety is attached to the first portion of the first target, wherein the third targeting moiety is attached to the second portion of the first target; forming a second probe complex including the second probe, the fourth targeting moiety, and the second target, wherein the second targeting moiety is attached to the first portion of the second target, wherein the fourth targeting moiety is attached to the second portion of the second target; separating the first probe complex and the second probe complex from the other components of the mixture; heating the first probe complex and the second probe complex; determining a first melting point of the first phase change material, wherein the first melting point is used to identify the first phase change material, wherein identifying the first phase change material is used to determine the first target; and determining a second melting point of the second phase change material, wherein the second melting point is used to identify the second phase change material, wherein identifying the second phase change material is used to determine the second target.

The above brief description of various embodiments of the present disclosure is not intended to describe each embodiment or every implementation of the present disclosure. Rather, a more complete understanding of the disclosure will become apparent and appreciated by reference to the following description and claims in view of the accompanying drawings. Further, it is to be understood that other embodiments may be utilized and structural changes may be made without departing from the scope of the present disclosure.

### BRIEF DESCRIPTION OF THE DRAWINGS

Many aspects of the disclosure can be better understood with reference to the following drawings.

The patent or application file contains at least one drawing executed in color. Copies of this patent or patent application publication with color drawing(s) will be provided by the Office upon request and payment of the necessary fee.

FIG. 1 illustrates SEM images of bare bismuth nanoparticles (A); TEM image (B), SEAD (C) and EDX spectrum (D) of encapsulated bismuth nanoparticles; the DSC curves of encapsulated bismuth nanoparticles (E) and encapsulated indium nanoparticles (F).

FIG. 2 illustrates the melting peaks (A) and the relation of peak width and temperature ramp rate (starting from the left, the top curve is at 10° C./min, the second curve down is 8° C./min, the third curve down is 5° C./min, the fourth curve down is 2° C./min, and the bottom curve is 0.5° C./min. (B) for encapsulated indium nanoparticles 1° C./min, the second curve down is 2° C./min, and the bottom curve is 5° C./min); the melting peaks (C) and the relation of peak width and the ramp rate 1.6 μM, the second curve down is 0.8 μM, the third curve down is 0.1 μM, and the bottom, curve is 0.05 μM) (D) for encapsulated bismuth nanoparticles; the melting peaks of encapsulated bismuth nanoparticles after the hybridization with target ssDNA (E); the relation between peak area and the concentrations of target ssDNA (F), and the DSC curve of encapsulated indium and bismuth nanoparticles collected in the same detection process (F inset).

FIG. 3A shows the DSC curve of lead-tin nanoparticles as a function of ssDNA concentration.

FIG. 3B shows the relation between peak area and target ssDNA concentration.

FIG. 4A shows the DSC curve of immobilized nanoparticles (diameter of 100 nm) as a function of avidin concentrations

FIG. 4B shows the relation between peak area and avidin concentration.

FIG. 5 illustrates Scheme 1 which shows thermally-addressed immunosorbent assay (TAISA).

FIG. 6 illustrates DSC curves: (A) and concentration dependent heat flows; (B) of indium nanoparticles that are immobilized on aluminum surfaces through biotin-avidin interaction, where the curves from up to down are at avidin concentrations of 20, 2, and 0.05 ng/ml, respectively; DSC curves (C) and concentration dependent heat flows; and (D) of lead-tin nanoparticles that are immobilized on aluminum surfaces through biotin-avidin interaction, where the curves from up to down are at avidin concentrations of 200, 20, 0.5, and 0.2 ng/ml, respectively.

FIG. 7 illustrates DSC curves: (A) of lead-tin nanoparticles at 200, 20, 2, and 0.5 ng/ml (top to down) of rabbit IgG; 20 ng/ml of human IgG does not cause attachment of nanoparticles (flat line); the relation between the heat flow (peak area) and rabbit IgG concentration (B), DSC curves (C) of tin nanoparticles that are immobilized at 2, 0.5, and 0.2 ng/ml (top to down) of human IgG; the relation between the heat flow and human IgG concentration; and (D) after immobilization of tin nanoparticles.

FIG. 8 illustrates a DSC curve of the multiplexed detection of 2 ng/ml of human IgG and 2 ng/ml of rabbit IgG using tin nanoparticles, and lead-tin nanoparticles, respectively (A); ramp rate dependent peak width for indium nanoparticles (B).

FIG. 9 illustrates DSC curves (A) and concentration dependent heat flow; (B) of lead-tin nanoparticles that are immobilized on aluminum surfaces by anti-rabbit-IgG and rabbit IgG interaction in diluted cell lysate, where rabbit IgG is intentionally added into the complex liquid at concentrations of 20, 10, and 2 ng/ml (top to down); (C) DSC curve of lead-tin nanoparticles that are immobilized by human IgG contained in the diluted cell lysate.

FIG. 10 illustrates TEM images of indium (A), lead-tin alloy (B), and tin (C) nanoparticles. FIG. 10 also illustrates: EDX (D) collected from surface-oxidized lead-tin nanoparticles; fluorescent images of protein covalently bounded onto surface-oxidized lead-tin nanoparticles (E) and the micro-patterns produced on a lead-tin thin film (F); EDX spectrum of an APTES modified aluminum surface (G); and fluorescent image of proteins covalently immobilized onto surface-modified micro-patterns (H).

FIG. 11 illustrates the simultaneous detection of multiple biomarkers using phase change nanoparticles.

FIG. 12 illustrates TEM images of indium (A), lead-tin alloy (B), tin (C) and bismuth (D) nanoparticles; XPS spectra of bismuth nanoparticles before (E) and after (F) sputtering with argon ions.

FIG. 13 illustrates the ramp rate dependent peak width for indium, bismuth, tin and lead-tin nanoparticles (A); melting time of 100 nm indium, lead-tin, tin and bismuth nanoparticles (B); DSC curves of 5 mg of indium nanoparticles at ramp rate of 1, 2, 5, 10, 20 and 50° C./min (C); ramp rate dependent peak area (D).

FIG. 14 illustrates the DSC curves of indium nanoparticles after detecting 10 nM ssDNA1 at different ratio of APTES and n-propyltriethoxysilane (1:021:1, 1:2, 1:5 and 1:10) (A); DSC curves of indium, bismuth, tin and lead-tin alloy nanoparticles after detecting ssDNA1 at concentration of 10 nM (B); DSC curves of indium nanoparticles after detecting 100 pM ssDNA1 at ramp rate of 50° C./min and bismuth nanoparticles after detecting 100 nM ssDNA2 at different ramp rate (1, 2, 5 and 10° C./min) (C); DSC curves of indium, lead-tin, tin and bismuth nanoparticles after detecting ssDNA1(100 nM, 10 nM, 1 nM and 100 pM), target ssDNA2 (100 nM, 10 nM, 1 nM and 100 pM), PSA (100, 20, 10 and 5 pg/ml), and human IgG (10, 2, 1 and 0.5 ng/ml), respectively.

FIG. 15 illustrates fluorescence microscopy images of indium (A) and (E), lead-tin (B) and (F), tin (C) and (F) bismuth (D) and (G) thin films with immobilized fluorescein isothiocyanate labeled bovine serum albumin (FITC-BSA) on APTES and n-propyltriethoxysilane modified silicon substrates, respectively.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of imaging, chemistry, synthetic organic chemistry, biochemistry, biology, molecular biology, microbiology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

As used herein, the term “host” or “subject” includes humans, mammals (e.g., cats, dogs, horses, etc.). Typical hosts to which embodiments of the present disclosure may be administered will be mammals (e.g., humans), particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. Target(s) from any of the foregoing can be detected using embodiments of the present disclosure.

The term “sample” can refer to a tissue sample, cell sample, a fluid sample, and the like. The sample may be taken from a host. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. Component(s) (target(s)) separated from the sample can be detected using embodiments of the present disclosure. In the present disclosure, the source of the sample is not critical.

The term “detectable” refers to the ability to detect a signal over the background signal.

The term “detectable signal” is a signal (thermal change) derived from the phase change material. The detectable signal is detectable and distinguishable from other background signals that may be generated. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

Phase change materials have unique thermophysical properties in that they can absorb thermal energy without temperature rise during the melting process. Once a state change occurs (e.g., solid changes to a liquid), heat is released or absorbed, and such a thermal change can produce a detectable signal that can be measured.

### General Discussion

Embodiments of the present disclosure provide for thermo-probes (also referred to herein as “probes”), particles including a plurality of probes, methods of making thermo-probes or particles, thermo-probe complexes, method of making thermo-probe complexes, methods of detecting a target, methods of detecting multiple targets, assays using the thermo-probes, immunoassays using the thermo-probes, methods of using thermo-probes or particles, and the like.

Embodiments of the present disclosure can detect a thermal signal that can be obtained by a phase change that is a characteristic of the nanoparticles including phase change material (e.g., thermo-probe or a particle as described herein). Embodiments of the present disclosure are advantageous in that they are very sensitive (e.g., below 8 nM). In addition, embodiments of the present disclosure allow for multiplexing so that multiple targets (e.g., from 10 s to 100 s up to about a 1000) can be detected during a single analysis. Further, in an embodiment, the particles, thermo-probes, and thermo-probe complexes can be analyzed multiple times in the same analysis and used multiple times in different analyses. In addition, the samples being analyzed do not need to have optical transparency to obtain a detectable signal.

The thermo-probe includes a phase change material. In an embodiment, the thermo-probe can be a nanoparticle made of a phase change material. In another embodiment, the thermo-probe can be a nanoparticle that includes an outer structure that encapsulates a phase change material. In a number of embodiments, the thermo-probe is a nanoparticle that includes an outer structure that encapsulates a phase change material; however, in each instance a nanoparticle made of a phase change material (also referred to as “phase change material nanoparticle”) without the outer structure can be used in an alternative embodiment.

In an embodiment, the thermo-probe includes a nanoparticle having an outer structure and an inner area. The outer structure encapsulates the inner area. In other words, the outer structure is a shell around the inner area. A phase change material is within the inner area. As a result, the outer structure is a shell around the phase change material. The melting point of the outer structure is greater than the melting point of the phase change material. The phase change material can change from a solid to a liquid upon exposure to heat while the outer structure remains a solid. The outer structure keeps the phase change material contained in the inner area so that it does not interfere with the analysis.

A targeting moiety can be attached (e.g., directly or indirectly) to the surface of the outer structure (or to the phase change material nanoparticle in an alternative embodiment), where the targeting moiety has an affinity for or binds with a target. Additional details regarding the targeting moiety and the target are described below.

In general, the thermo-probe has a spherical cross-section or substantially spherical cross-section (e.g., about 70, 80, or 90% relative to a spherical cross-section); however, embodiments of the present disclosure encompass probes having different cross-sections.

In an embodiment, the outer structure can be composed of a material such as silica, alumina, titania, polymer, an oxide of the phase change material (as long as the oxide has a high enough melting point to be used in the particular embodiments), or a combination thereof. In an embodiment, the outer structure can be made by thermo-decomposition of precursors of the phase-change material, polymers, and/or surfactants. In an embodiment, the outer structure is silica. The outer structure can have a thickness of about 2 nm to 200 nm or about 5 nm to 100 nm. The outer structure can have a diameter (or the largest distance from one side of the probe to the other side of the probe) of about 1 nm to 5000 nm, about 1 nm to 1000 nm, about 10 nm to 1000 nm, about 10 nm to 500 nm, or about 10 to 250 nm.

In an embodiment where the outer structure is silica, the silica outer structure can be made by thermally decomposing tetraethylorthosilicate at about 70° C. Additional details are provided in the Examples.

As noted above, one or more targeting moieties (same type of targeting moiety or different types) can be attached (e.g., directly or indirectly (e.g., using a linker)) to the outer surface (or to the phase change material nanoparticle in an alternative embodiment). The number of targeting moieties attached to the outer surface can be about 1 to 1000 and for larger probes more than 1000. In an embodiment, different types of targeting moieties that are directed to different portions of the target can be attached to the outer surface. In an embodiment, different types of targeting moieties that are directed to different targets can be attached to the outer surface.

The targeting moiety (e.g., first, second, third, etc, targeting moieties) has an affinity (e.g., an attraction to) for or can bind (e.g., chemical bonding, covalently or ionically), biological interaction, biochemical interaction, and the like) to a target (e.g., a compound, a cell, a tissue, a protein, an antibody, an antigen, and the like). The targeting moiety can include, but is not limited to, polypeptides (e.g., proteins such as, but not limited to, antibodies (monoclonal or polyclonal)), antigens; nucleic acids (both monomeric and oligomeric), DNA (ss or ds), polysaccharides, sugars, fatty acids, steroids, purines, pyrimidines, ligands, or combinations thereof. In an embodiment, the targeting moiety can have an affinity for or bind to one or more targets.

The targeting moiety can be attached or bind, directly and/or indirectly (e.g., using one or more linker compounds) to the outer structure, or to a compound or a coating disposed on the outer structure (the phase change material nanoparticle in an alternative embodiment). In an embodiment, the targeting moiety can be attached directly or indirectly to the outer structure via chemical bonding (e.g., covalently or ionically), biological interaction, biochemical interaction, and/or otherwise associated with the outer structure. In particular, the targeting moiety can be attached directly or indirectly to the outer structure via a covalent bond, a non-covalent bond, and/or an ionic bond, as well as being attached through interactions such as, but not limited to, chelation interaction, hydrophobic interaction, hydrophilic interaction, charge-charge interaction, π-stacking interaction, combinations thereof, and like interactions.

As mentioned above, phase change materials have unique thermophysical properties. Once a state change occurs (e.g., solid changes to a liquid), heat is released or absorbed, and such a thermal change can produce a detectable signal that can be measured. As a result, phase change materials can be designed and selected based on their melting points. Thus, probes can be designed so that each probe includes a phase change material that has a melting point that is different than the other probes. As a result, multiplex detection can be performed using a number of probes where each probe has a different phase change material.

Phase change material can be a solid-to-solid, a solid-to-liquid, a solid-to-gas, or a liquid-to-gas, phase change material. The types of phase change material can include materials that can undergo any of solid-to-solid, a solid-to-liquid, a solid-to-gas, or a liquid-to-gas, phase changes. In an embodiment, the phase change material is a solid-to-liquid phase change material. In an embodiment, the phase change material is a reversible phase change material so the probe can be heated and cooled multiple times in the same detection analysis and/or used multiple times in different detection analyses. The phase change material can be organic, inorganic, a eutectic alloy, an alloy, or a combination thereof. The phase change material can be selected from: indium (e.g., indium acetate is a precursor material), tin (e.g., tin acetate is a precursor material), lead (e.g., lead acetate is a precursor material), bismuth (e.g., bismuth acetate is a precursor material), gold, silver, salt (NaCl, etc), paraffin wax, and other organic materials can also be used. Eutectic alloys that can be used include Ag (silver), Al (aluminium), Au (gold), Bi (bismuth), Cu (copper), In (indium), Ni (nickel), Pb (lead), Sb (antimony), Sn (tin), Zn (zinc), and other elements. Alloys that can be used include a binary, ternary, or other higher ordered alloys of the elements that can from alloys. Organic materials that can be used include paraffin wax, organic solid, organic acids, and the like. Although some specific phase change materials are described, it is contemplated that other phase change materials can be used as long as they act in a manner consistent with the teachings of the present disclosure.

The amount of phase change material in the inner area will depend upon the area of the inner area as well as the volumetric expansion characteristics of the phase change material. The probe can be designed to accommodate volumetric expansion characteristics of the phase change material and the amount of phase change material in the inner area.

As mentioned above, in an alternative embodiment the nanoparticle is a phase change material nanoparticle that does not include an outer structure and the phase change materials described herein also describe the phase change material that can be used in this embodiment.

The melting point of the phase change material depends upon the specific phase change material. In an embodiment, the melting point can be about 50 to 1000° C. or about 100 to 700° C. In detection analyses that use multiple probes, the separation between melting points can be 1° C. or more to obtain a detectable signal. In many instances, 2° C. of separation is enough to separate the melting points to obtain a detectable signal. In some embodiments the separation between melting points can be 0.5° to 1° C.

Embodiments of the thermo-probe can be used to form a thermo-probe complex. Additional details regarding detecting a target using a probe and probe complex are described hereafter; however the following discussion is directed to the thermo-probe complex.

An embodiment of a thermo-probe complex can include a probe, a capture moiety, and a target. The probe or thermo-probe is described above. In an alternative embodiment, the thermo-probe is a phase change material nanoparticle that does not include an outer structure. The first targeting moiety of the thermo-probe complex can attach or bind to a first portion of the target. The capture moiety includes a second targeting moiety that can attach or bind to a second portion of the target. The first portion and the second portion of the target are different.

In short, the thermo-probe complex includes three portions: the thermo-probe attached to the target, and the target attached to the capture moiety. Specifically, the thermo-probe complex includes the thermo-probe, where the first targeting moiety is attached to the first portion of the target. The second portion of the target is attached to the second targeting moiety of the capture moiety.

As mentioned above, the target can include, but is not limited to, a compound, a polypeptide, a nucleic acid, a polysaccharide, a sugar, a fatty acid, a steroid, a purine, a pyrimidine, a hapten, a ligand, a cell type, a cell surface, extracellular space, intracellular space, a tissue type, a tissue surface, vascular, and the like. The targeting moiety can be selected based on the target.

As noted above, the target has, a first portion and a second portion, where the first portion and the second portion are not the same. One of the probe or the capture moiety can bind with one of the first portion or the second portion, while the other of the probe and the capture moiety can bind to the other of the first portion or the second portion (e.g., the probe binds to the first portion and the capture moiety binds to the second portion).

As noted above, the capture moiety can include the second targeting moiety. The second targeting moiety can be any of the targeting moieties described above. The capture moiety includes a structure or substrate that is attached (e.g., in any of the ways described above for attaching or binding) to the second targeting moiety.

In an embodiment, the structure can include a magnetic structure. The magnetic structure of the capture moiety (also referred to as “magnetic capture moiety”) can be used to separate the probe complex from other components in a mixture using a magnetic separation system (e.g., iron oxide, cobalt, and nickel). The magnetic structure can include nanoparticles and/or microparticles. The magnetic structure can have a diameter of about 5 nm to 10 μm. The magnetic structure can include compounds or be coated with a material so that the second targeting moiety can be attached to the magnetic structure.

In an embodiment, the substrate of the capture moiety (also referred to as the “capture moiety structure”) can include one second targeting moiety. In another embodiment, the substrate can include multiple second targeting moieties (e.g., multiple sites for the targeting moiety to attach). The targeting moieties can be the same type or of different types (e.g., a second, third, fourth, etc, targeting moiety) for multiplexing. The substrate is similar to an array having multiple addressable sites. In an embodiment, the substrate can be an aluminum substrate, gold, ceramic, glass, silicon dioxide, and silicon, etc. In an embodiment; the substrate can include compounds or be coated with a material to attach to the targeting moiety.

In an embodiment, a plurality of nanoparticles (as described in reference to the probes) can be within a particle, such as a microparticle (e.g., a diameter (or longest dimension) of about 500 nm to 10 μm). In an embodiment, the nanoparticles are enclosed by a particle shell structure (e.g., a thickness of about 1 to 100 nm). The particle shell structure can be made of a material such as silica, alumina, titania, a polymer, or a combination thereof. In an embodiment, the particle shell structure is hollow if the nanoparticles are not disposed within the particle shell structure of the particle, while in another embodiment the particle shell structure fills in the voids not occupied by the nanoparticles within the particle.

In general, the particle has a spherical cross-section or substantially spherical cross-section (e.g., about 70, 80, or 90% relative to a spherical cross-section); however, embodiments of the present disclosure encompass a particle having different cross-sections.

The number of nanoparticles that can be within the particle can be about 2 to 50 nanoparticles. The nanoparticles can each be of the same type (e.g., have the same phase change material within the nanoparticle), each being a different type (e.g., have the different phase change material within the nanoparticle), or a mixture thereof.

Embodiments of the particles can be used as thermal barcodes since the selected number and/or types of phase change materials used in each of the nanoparticles can produce on the order of 10⁹ or more unique thermal signatures. In an embodiment, a thermal barcode can also be used to label cells, cancer cells, and any non-biological objects.

Embodiments of the thermo-probe complex and thermo-probe can be used to detect one or more targets. An embodiment of a method includes mixing a probe, a capture moiety, and a target, each of which are described herein. The probe can be a nanoparticle having an outer surface around the phase change material, or in an alternative embodiment, the nanoparticle can be a phase change material nanoparticle without an outer structure. The target can be in a sample such as a bodily fluid or from another source. Although only one target is described in this embodiment, the sample may include multiple components including multiple different targets. Also, the sample may not include the specific target of interest. For clarity, the following embodiment includes a target so that this embodiment can be described. Other embodiments including multiple targets are described below.

The thermo-probe, capture moiety, targeting moieties, and target, can be any of those noted above. The probe includes a first targeting moiety and the capture moiety includes a second targeting moiety. The first targeting moiety has an affinity for a first portion of the target. The second targeting moiety has an affinity for a second portion of the target. As noted above, the first portion of the target and the second portion of the target are not the same. Once the probe, capture moiety, and target are mixed, a probe complex including the probe, the second targeting moiety, and the target can be formed. The first targeting moiety is attached to the first portion of the target and the second targeting moiety is attached to the second portion of the target.

Subsequently, the thermo-probe complex can be separated from the other components of the mixture. In an embodiment, the separation can be conducted using a magnetic system if the capture moiety is a magnetic capture moiety. In particular, the magnetic system can attract the probe complex while the other components are removed or rinsed away. Alternatively, the probe complex can be removed from the other components.

In another embodiment, the separation can be performed by removing or rinsing the other components from the structure of the capture moiety. Once the other components are removed, the probe complex can be removed from the structure or the probe complex can be left attached to the structure. In an embodiment, the structure can include multiple sites for the second targeting moiety.

Once the thermo-probe complex is separated from the other components, the probe complex can be heated. In an embodiment, the rate of temperature increase can be about 0.2 to 20° C. per minute. The rate of temperature increase can be selected based on the phase change material(s) being used. The heating and temperature measurements can be conducted using differential thermal analysis.

Once the melting point is determined, the melting point is used to identify the phase change material since each phase change material to be used in a detection system would have a different and distinguishable melting point. Said another way, the melting point for the phase change material is known, so once the melting point is known, it can be correlated to the phase change material. Once the phase change Material is determined, the identity of the target can be determined since each probe will have a targeting moiety for a specific target. Although only one probe is noted above, a plurality of probes can be used and the probe (or probes) that attaches to the target can be determined based on the melting point, which is then used to determine the target. This can be used to quantify the amount of target by measuring the heat flow, the mass of phase change materials, and the grafting density of target on the nanoparticles. Thus, embodiments of the present disclosure can detect the presence of a target and the concentration of the target in the sample.

In another embodiment, the multiple targets can be detected using thermo-probes and thermo-probe complexes. The method includes mixing a first probe, a second probe, a first capture moiety, a first target, and a second target, each of which are similar to the thermo-probe, capture moiety, and target mentioned above. Unlike the embodiment that follows this embodiment, this embodiment uses a single capture moiety, while the following embodiment uses two capture moieties.

The first thermo-probe includes a first phase change material having a first melting point, while the second thermo-probe includes a second phase change material having a second melting point. The first melting point and the second melting point are detectably different and distinguishable. The first thermo-probe includes a first targeting moiety, while the second thermo-probe includes a second targeting moiety. The first capture moiety includes a third targeting moiety. The first targeting moiety has an affinity for a first portion of the first target. Each of the first, second, and third targeting moieties can be similar to the targeting moieties noted above. The third targeting moiety has an affinity for a second portion of the first target. The first portion of the first target and the second portion of the first target are not the same. The second targeting moiety has an affinity for a first portion of the second target. The third targeting moiety has an affinity for a second portion of the second target in addition to the second portion of the first target. The first portion of the second target and the second portion of the second target are not the same. In an embodiment, the second portion of the first target and the second target are the same, while in another embodiment they are different.

Once the first thermo-probe, the second thermo-probe, the first capture moiety, the first target, and the second target are mixed, a first thermo-probe complex and a second thermo-probe complex are formed. The first thermo-probe complex includes the first probe, the third targeting moiety, and the first target. The first targeting moiety is attached to the first portion of the first target, while the third targeting moiety is attached to the second portion of the first target. The second thermo-probe complex includes the second probe, the third targeting moiety, and the second target. The second targeting moiety is attached to the first portion of the second target, while the third targeting moiety is attached to the second portion of the second target.

The first thermo-probe complex and the second thermo-probe complex can be separated from the other components of the mixture in a similar manner as described above. Subsequently, the first probe complex and the second probe complex can be heated. The heating can be conducted in a similar manner as described above to determine the first and the second melting points.

Once the first and the second melting points are determined, the first and second melting points can be used to identify the first and second phase change materials since each phase change material to be used in a detection system would have a different and distinguishable melting point. Said another way, the melting points for the first and second phase change materials are known, so once the melting points are known, they can be correlated to the first and second phase change material. Once the first and the second phase change material are determined, the identity of the first and second targets can be determined since each of the first and second probes will have a targeting moiety for a specific target (first and second targeting moiety). Although only two probes are noted above, a plurality of probes can be used and the probe (or probes) that attaches to the target(s) can be determined based on the melting point(s), which is then used to determine the target(s). This method can be used to derive the amount of targets by measuring the heat flow, the mass of phase change nanoparticles, and the latent heat of fusion of the phase change materials.

In another embodiment, multiple targets can be detected using thermo-probes and thermo-probe complexes. The probe can be a nanoparticle having an outer surface around the phase change material, or in an alternative embodiment, the nanoparticle can be a phase change material nanoparticle without an outer structure. The method includes mixing a first probe, a second probe, a first capture moiety, a second capture moiety, a first target, and a second target, each of which are similar to the probe, capture moiety, and target mentioned above.

The first thermo-probe includes a first phase change material having a first melting point, while the second probe includes a second phase change material having a second melting point. The first melting point and the second melting point are detectably different and distinguishable. The first probe includes a first targeting moiety, while the second probe includes a second targeting moiety. The first capture moiety includes a third targeting moiety. The second capture moiety includes a fourth targeting moiety. Each of the first, second, third, and fourth targeting moieties can be similar to the targeting moieties noted above.

The first targeting moiety has an affinity for a first portion of the first target. The third targeting moiety has an affinity for a second portion of the first target. The first portion of the first target and the second portion of the first target are not the same.

The second targeting moiety has an affinity for a first portion of the second target. The fourth targeting moiety has an affinity for a second portion of the second target. The first portion of the second target and the second portion of the second target are not the same.

Once the first thermo-probe, the second thermo-probe, the first capture moiety, the second capture moiety, the first target, and the second target are mixed, a first thermo-probe complex and a second thermo-probe complex are formed. The first thermo-probe complex includes the first probe, the third targeting moiety, and the first target. The first targeting moiety is attached to the first portion of the first target, while the third targeting moiety is attached to the second portion of the first target. The second thermo-probe complex includes the second probe, the fourth targeting moiety, and the second target. The second targeting moiety is attached to the first portion of the second target, while the fourth targeting moiety is attached to the second portion of the second target.

The first thermo-probe complex and the second thermo-probe complex can be separated from the other components of the mixture in a similar manner as described above. Subsequently, the first thermo-probe complex and the second thermo-probe complex can be heated. The heating can be conducted in a similar manner as described above to determine the first and the second melting points.

Once the first and the second melting points are determined, the first and second melting points can be used to identify the first and second phase change materials since each phase change material to be used in a detection system would have a different and distinguishable melting point. Said another way, the melting points for the first and second phase change materials are known, so once the melting points are known, they can be correlated to the first and second phase change material. Once the first and the second phase change material are determined, the identity of the first and second targets can be determined since each of the first and second probes will have a targeting moiety for a specific target (first and second targeting moiety).

Although only two probes are noted above, a plurality of probes can be used and the probe (or probes) that attaches to the target(s) can be determined based on the melting point(s), which is then used to determine the target(s). This method can be used to derive the amount of targets by measuring the heat flow, the mass of phase change nanoparticles, and the latent heat of fusion of the phase change materials.

It should be noted that various parameters, such as diameter, thickness, temperature, and the like, have been expressed as one or more values or ranges of values, but it is contemplated that one of skill in the art would recognize that the values of one or more of the parameters may extend beyond those values or ranges described herein if the resultant probe, probe complex, particle, or the like, still operates and has the characteristics as described herein.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Brief Introduction

We describe a multiplexed highly sensitive method to detect multiple cancer biomarkers using silica encapsulated phase change nanoparticles as thermal barcodes. During phase changes, nanoparticles absorb heat energy without much temperature rise, and show sharp melting peaks (0.6° C.). A series of phase change nanoparticles of metals or alloys can be synthesized in such a way that they melt between 100-700° C., thus the multiplicity could reach 1000. The method has high sensitivity (8 nM) that can be enhanced using material with large latent heat, nanoparticles with large diameter, or reducing the grafting density of biomolecules on nanoparticles.

### Introduction

Solid to liquid phase change materials (PCM) have unique thermophysical properties. PCM can adsorb thermal energy without temperature rise during the melting process. As long as the solid phase exists, all adsorbed thermal energy is used to melt solid phase, and the theoretical peak width is zero (Ind. Eng. Chem. Res. 43, 5350 (2004)). In practice, the heat transfer from the surrounding environment into the core of PCM takes time, and such delay in melting broadens the melting peak because the temperature scan is done at a constant rate (0.02 to 20° C. per minute). At normal condition, the peak width is less than 1° C. All materials exhibit reversible solid-liquid transitions at certain temperature. The melting temperatures depend on atomic numbers (pure metals) and compositions (alloys) if the diameter of PCM materials is larger than critical sizes, where surface atoms play an important role in decreasing melting temperatures (J. Appl. Phys. 97, 034304 (2005)). In addition, a variety of alloys melt in the operating range of thermal analysis equipment (100-700° C.) (Appl. Thermal Eng. 23, 251 (2003)), and PCM can be encapsulated inside shells to prevent the changes in compositions (Int. J. Energy Res. 26, 159 (2002)).

We have developed a multiplexed highly sensitive method to detect cancer biomarkers (i.e., oligonucleotides), where silica encapsulated phase change nanoparticles of metals are used as thermal barcodes. The silica shells do not melt due to its high melting temperature (1650° C.). The thermal signals are readout by differential scanning calorimetry (DSC), where the melting temperatures and the latent heats of fusion are used to determine the existence and concentrations of biomarkers. The major steps of this method are (1) the immobilization of single strand DNA (ssDNA1) on silica shells of encapsulated PCM nanoparticles; (2) the immobilization of another ssDNA (ssDNA2) on magnetic nanoparticles; (3) the hybridization of target ssDNA at two ends with ssDNA1 and ssDNA2, which will connect encapsulated PCM nanoparticles and magnetic nanoparticles; (4) the magnetic separation of unhybridized target ssDNA and ssDNA1; (5) the thermal readout of PCM nanoparticles. The sequence of ssDNA1 is 5′-TTA CAA TAA TCC ATT ATT ATT A/3ThioMC3-D-3′; that of target ssDNA is 5′-GAA TTA TTG TAA ACA CAG CAA CCA CAT-3′; and that of ssDNA2 is 5′-/5Biosg/ATG TGG TTG CTG TGT-3′. These sequences are designed randomly and are representative for any genetic biomarkers. Instead of making every encapsulated PCM nanoparticles that have melting temperatures covering entire thermal scan range, the main purpose of this paper is to prove the concept of encapsulated PCM nanoparticles as thermal barcodes for the detection of biomarkers.

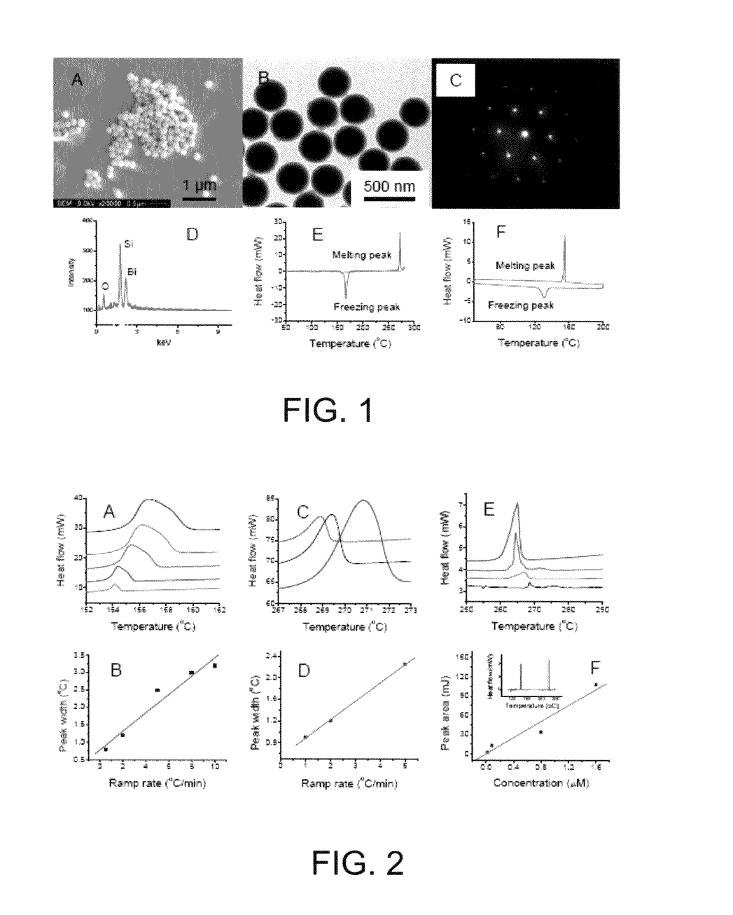

The PCM nanoparticles are synthesized by the thermal decompositions of organometallic precursors. The precursor (bismuth acetate or cyclopentadienyl indium) is dissolved in ethylene glycol in presence of poly-vinylpyrrolidone (PVP) (Angew. Chem. Int. Ed. 40, 448 (2001) and Chem. Mater. 20, 3656 (2008)). Taking bismuth nanoparticle as example, 0.75 mmol bismuth acetate is added into 20 ml ethylene glycol with 0.2 g PVP. The mixture is heated to 200° C. in nitrogen. The reaction is quenched after 20 min by pouring the mixture into 200 ml ethanol pre-cooled to 0° C. After centrifuging and washing the nanoparticles, the thermal decomposition of tetraethoxysilane (TEOS) is used to generate silica, where 3.5 ml ammonia hydroxide (28%) and 0.2 ml TEOS are added into a 50 ml ethanol that contains 50 mg suspended nanoparticles (J. Mater. Chem. 14, 966 (2004)). The mixture is sonicated at 70° C. for 1 hr, followed by centrifuging and washing by ethanol. FIG. 1A is a scanning electron microscopy (SEM) image of bismuth nanoparticles. The diameters can be controlled from 100 to 500 nm by changing reaction time and amounts of reactants. These nanoparticles are coated with ˜20 nm silica as shown in transmission electron micrograph (TEM) (FIG. 1B). The nanoparticles at room temperature have crystalline structure as shown in selected area electron diffraction (SAED) pattern (FIG. 1C). Energy dispersive X-ray (EDX) analysis confirms the compositions of core and shell materials (FIG. 1D), where the signals of silicon and bismuth can be seen clearly. The solid to liquid phase changes are studied by using differential scanning calorimetry (DSC), which is operated from 50 to 300° C. at ramp rate of 2° C./min. The DSC curves of encapsulated bismuth and indium nanoparticles show single peak with full width at half height of less than 1° C. (FIGS. 1E and 1F), where the two peaks in each figure correspond to the melting and freezing of PCM cores as temperature increases and decreases. Although nanoparticles with diameter smaller than the critical diameter (10 nm) melt at lower temperatures, encapsulated nanoparticles made here have similar melting temperature as bulk materials due to their larger diameters (J. Am. Chem. Soc. 124, 2312 (2002)). In addition, supercooling of molten metals occurs in both cases when the temperature is reduced at the same rate.

The procedures of detecting genetic biomarker using encapsulated PCM nanoparticles are summarized as following. The silica shell is first modified with amine-terminated monolayer by adding aminopropyltriethoxysilane to the suspension of encapsulated nanoparticles in toluene. After reacting for 3 hrs at room temperature, extra silane molecules are removed by centrifuging. The nanoparticles are resuspended in anhydrous dimethyl sulfoxide that contains N-succinimidyl (4-iodoacetyl) aminobenzoate (SIAB) and incubated to complete the reaction. After removing excess SIAB by centrifuging, nanoparticles are incubated with 3′ thiolated ssDNA (ssDNA1) in a buffer (pH 8.0) at room temperature (Anal. Chem. 69, 2438 (1997)). The ssDNA1 is complementary to one end of the target ssDNA. In order to modify magnetic nanoparticles with separating ssDNA (ssDNA2) which is complementary to the other end of the target ssDNA, avidin is conjugated with polyacrylic-acid-coated iron oxide nanoparticles (10 nm diameter). The suspension of avidin-modified magnetic nanoparticles is incubated with biotinylated oligonucleotides in a buffered solution (pH 7), and unbound ssDNA2 is removed by dialysis. The hybridization is done by incubating target ssDNA with excess amount of ssDNA1 modified PCM nanoparticles, and ssDNA2 modified magnetic nanoparticles in a buffer solution (pH of 7) at 37° C. After hybridization, the encapsulated PCM nanoparticles with unhybridized ssDNA1 are removed using an NdFeB magnet; and the aggregates formed by the PCM nanoparticles and magnetic particles are transferred into aluminum pans and analyzed using a Perkin-Elmer DSC (DSC 7).

The melting peaks of encapsulated PCM nanoparticles are used for biomarker detections. For encapsulated indium nanoparticles, the width of melting peak become smaller as the ramp rate of temperature decreases (FIG. 2A). A linear relation exists between the ramp rate and the peak width (FIG. 2B), where the smallest peak width among the studied ramp rates is −0.6° C. The peak areas change with the ramp rates, probably due to the heat loss during heating process. In the case of encapsulated bismuth nanoparticles, the peak widths also depend on the ramp rate (FIG. 2C), and the relation between the ramp rate and the peak width is also linear (FIG. 2D). The similarity in both cases suggests that the peak width is mostly determined by the temperature ramp rate. At the peak width of 0.6° C., the number of thermal barcodes could reach 1000 from 100 to 700° C. FIG. 2E shows the melting curves of four aggregates of encapsulated bismuth nanoparticles and iron oxide nanoparticles formed after DNA hybridization. Although iron oxide does not melt during the thermal scan (melting temperature over 1000° C.), it can change the heat transfer into PCM nanoparticles because of its low thermal conductivity, thus the melting peaks become asymmetric. As the results, the melting peaks shift towards high temperature side slight, especially at low target ssDNA concentrations, where the ratio of PCM nanoparticles to magnetic nanoparticles is low. The area of melting peak is proportional to the mass of PCM nanoparticles, and can be obtained by integrating the heat flow over the melting range. A linear relation exists between the heat fluxes and the target ssDNA concentrations (FIG. 2F), from which the lowest detectable concentration of target ssDNA is 8 nM. The detection sensitivity depends on the latent heat of PCM and the diameter of PCM nanoparticles, as well as the grafting density of ssDNA on PCM nanoparticles. For encapsulated nanoparticles of bismuth and indium, the areas of melting peaks are different even if the masses and the diameters of these nanoparticles are close to each other. At last, the encapsulated indium and bismuth nanoparticles are mixed at roughly the same concentrations, modified with ssDNA1, and used to detect target ssDNAs. The DSC curve shows two peaks with melting temperatures at 155 and 271° C., which correspond to the melting peaks of indium and bismuth, respectively. The peak area (i.e., heat flux) of indium is smaller than that of bismuth, because the latent heat of indium (28 J/g) is smaller than that of bismuth (52 J/g). The ratio of integrated peak area (0.61) is close to the ratio of latent heats of indium and bismuth (0.54), meaning the chances of ssDNA immobilizations, and hybridizations on these two types of nanoparticles are close to each other.

The minimal detectable heat flux in the commercial DSC is determined by its root mean square (RMS) noise (0.2 μW), which corresponds to an energy flow of 0.2 μJ for 1° C. wide peak at a ramp rate of 1° C./second. If bismuth nanoparticles of 200 nm diameter are used, the number of nanoparticles that can adsorb 0.2 μJ heat is 9×10⁴. Providing one DNA double helix is formed on each nanoparticle in a 1 ml solution, the according concentration of target ssDNA is 1.5×10⁻¹⁶ M or 0.15 fM, which is lower than most of current detection methods. The current detection limit is not as good as the theoretical one, probably because thousand of target ssDNA are attached on each nanoparticle. The detection sensitivity can be enhanced by using larger diameter particles, materials with large latent heats of fusion or reducing the grafting density. Microcalorimetry can measure the formation enthalpy of DNA duplex (Biopolymers 87, 293 (2007)), but it cannot detect genetic biomarkers due to the sequence-independent enthalpy change. The proposed method works for turbid samples, and is immune to colored molecules, salts, or conductive materials, which do not change phase in the operating range. The interferences from organic carbon can be excluded by preheating samples. The detection capacity and analysis time can be optimized by changing ramp rate. In the case of fast scan, the temperature ramp rate can be 50° C. per min, and an entire thermal scan from room temperature up to 1000° C. takes only 20 min. Due to wide ranges of material choices, a one-to-one correspondence can be established between the melting temperatures of PCM nanoparticles and types of biomarkers, allowing easy readout of thermal signals. At last, detecting biomarkers in thermal space provides an alternative that is independent of the optical, electric, magnetic and electrochemical properties of nanoparticles. The large thermal scan range, sharp melting peak, large latent heat, and diverse material choice pave the way to detect multiple low concentration not-effective biomarkers using encapsulated phase change nanoparticles at early stage of cancers.

### Example 2

### (1) DNA Detection on Solid Substrate

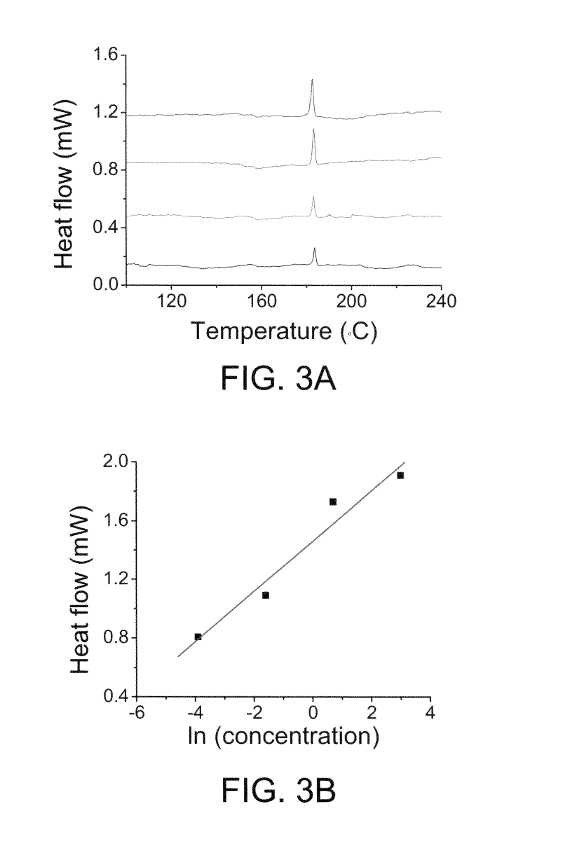

Not only magnetic nanoparticles can be used for detecting DNA, the capture ssDNA can be immobilized on solid substrates to detect target ssDNA. We have modified an aluminum surface using APTES, and immobilized capture ssDNA on the surface. The target ssDNAs dissolved in phosphate buffers at pH 4.5 are loaded at different concentration. The probe ssDNA modified nanoparticles, and the capture ssDNA modified aluminum surface are added into the solution for DNA hybridization. After hybridizing for 30 minutes, the aluminum surface is taken out of the solutions, washed by phosphate buffer and tested by DSC. The samples that contain a variety of concentrations of target ssDNAs (8 nM to 1 pM) in 1M NaCl and tris-EDTA are studied. The melting peaks of DNA immobilized nanoparticles are used for the qualitative and quantitative detections of DNA biomarkers. FIG. 3A shows melting peaks of lead-tin nanoparticles, where the curve from the top to bottom corresponds to target ssDNA concentration from 10 nM to 0.5 pM. Since the melting temperature of nanoparticles is much higher than those of species contained in bloods, the melting peak is assigned to biomarkers. The area of melting peak is proportional to the mass of PCM nanoparticles, and can be obtained by integrating the heat flow over the melting range. FIG. 3B shows the relation between peak area and target ssDNA concentration. A linear relation exists between target ssDNA concentration and peak area at relatively high concentrations. The curve shows a relatively large heat flow at lower concentration, probably due to residual nanoparticles in the solution that are not washed away.

### (2) Protein Detection Using Thermal Nanoparticles

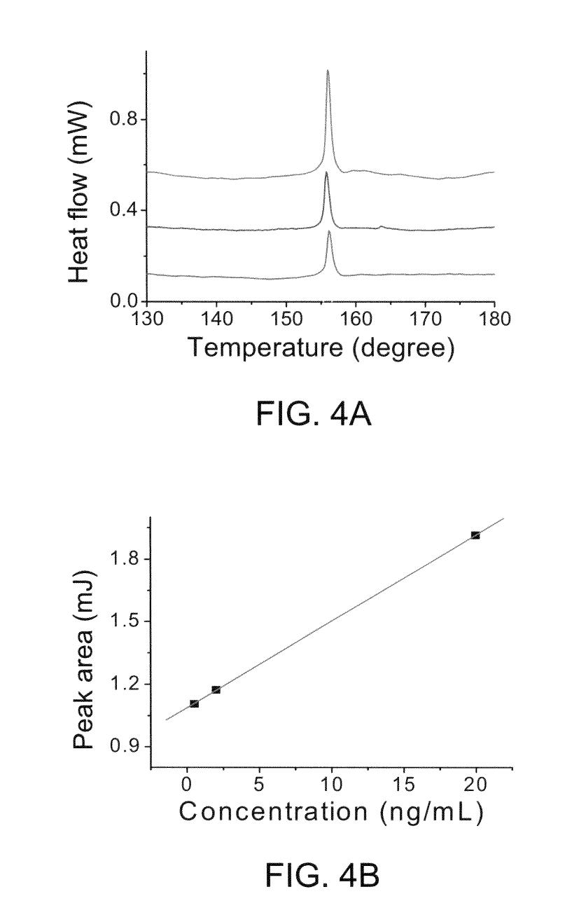

The thermal immunoassay is used to detect the concentrations of avidin. The amine-modified tin nanoparticles and aluminum plate are conjugated with biotins by incubating both of them with 3% carboxyl-terminated long chain biotins in anhydrous toluene for 10 hours. After modification, tin nanoparticles and aluminum plate are washed for three times to remove physically adsorbed biotins. XPS and fluorescence microscope confirm the outcomes of biotin conjugation. The biotin-avidin conjugation is then carried out by incubating biotin modified tin nanoparticles and aluminum plate inside the solution that contains avidin at certain concentration for 30 minutes. The unconjugated nanoparticles are rinsed away, and the aluminum plate is imaged by scanning electron microscope (SEM), where tin nanoparticles are immobilized on the aluminum surface as a monolayer. Increasing the amount of avidin leads to larger amount of immobilized nanoparticles. The DSC experiments are performed from 50 to 300° C. with the temperature ramp rate of 10° C./min. FIG. 4A is the DSC curve of immobilized nanoparticles (diameter of 100 nm) as a function of avidin concentrations, where nanoparticles melt at 230° C., and the heat fluxes decrease linearly as the concentration of avidin is reduced to 10 ng/ml. The lowest detectable concentration of avidin is determined to be 8 nM by extrapolating the linear portion of the concentration versus heat flux curve (FIG. 4B). Such detection limit is lower than those of available optical detection methods.

### Example 3

### Brief Introduction

Thermally-addressed immunoassay is developed to detect multiple proteins using phase change nanoparticles as thermal barcodes. The solid to liquid phase changes of nanoparticles adsorb heat energy and generate sharp melting peaks, which are used as thermal signatures to determine the existence and concentration of proteins. Multiple proteins can be detected by using different types of nanoparticles in order to create a one-to-one correspondence between one type of nanoparticles and one type of protein. The fusion enthalpy that is proportional to the amount of phase change materials has been used to derive the amount of protein. The melting temperatures of nanoparticles are designed to be higher than 100° C. to avoid interference from species contained in the fluid. Thus, the use of thermal nanoparticles allows the detection of multiple low concentration proteins in a complex fluid such as cell lysate regardless of the color, salt concentration, and conductivity of the sample.

### Introduction:

Detecting protein biomarkers in complex fluids such as blood, urea or cell lysate offers great benefits in sample preparation and target retention for early disease detection (Curr. Opin. Mol. Ther. 2007, 9, 563; J. Natl. Cancer Inst. 2004′, 96, 353; Nature 2008, 452, 548). Enzyme-linked immunosorbent assay (ELISA) relies on antigen-antibody interaction to detect antibodies (i.e., proteins), and the binding events are readout through enzyme activities, or the physical, chemical or spectroscopic labels attached on the antibodies at different sites (Nanotechnology 2008, 19, 375502; Science 2003, 301, 1884; Nature Rev. Cancer 2005, 5, 161). Owing to the small sizes, nanoparticles have intimate contacts with target proteins in solutions, and have been used to detect specific'antigen-antibody interaction (Anal. Chem. 2006, 78, 2055; Chem. Rev. 2005, 105, 1547; Nature Biotechnol. 2004, 22, 47; Nature Biotechnol. 2001, 19, 631), where the sensitivity is enhanced by amplifying the physical signatures of nanoparticles. Although widely used, ELISA has some limits: (1) it requires extensive efforts and expensive agents to produce testing samples that are free of colored species, suitable pH and salt concentration; species that interfere with the activity of enzyme should be removed; (2) the multiplicity is often low, even if nanoparticles are used as probes, which makes it hard to detect multiple proteins from a small amount of sample. In this aspect, although optical absorbances of metallic and semiconductor nanoparticles can be controlled in the range of 400-900 nm by changing diameters (J. Am. Chem. Soc. 2006, 128, 2526), the absorbance or emission peaks in this region are wide (−150 nm) that limits the number of biomarkers that can be detected in one assay due to peak overlap (Curr. Opin. Biotechnol. 2002, 13, 40). Metal or magnetic nanoparticles cannot be used to detect multiple proteins, because different nanoparticles are not distinguishable by their electric or magnetic properties (Angew. Chem. Int. Ed. 2001, 40, 3204; Angew. Chem. Int. Ed. 2000, 39, 3845; J. Am. Chem. Soc. 2007, 129, 11356; Nature Biotechnol. 2005, 23, 1294). Electrochemical method can detect few types of nanoparticles, but the peaks are wide and voltage range is narrow (few voltages), which limits its detection multiplicity (Anal. Chem. 2001, 73, 5576).

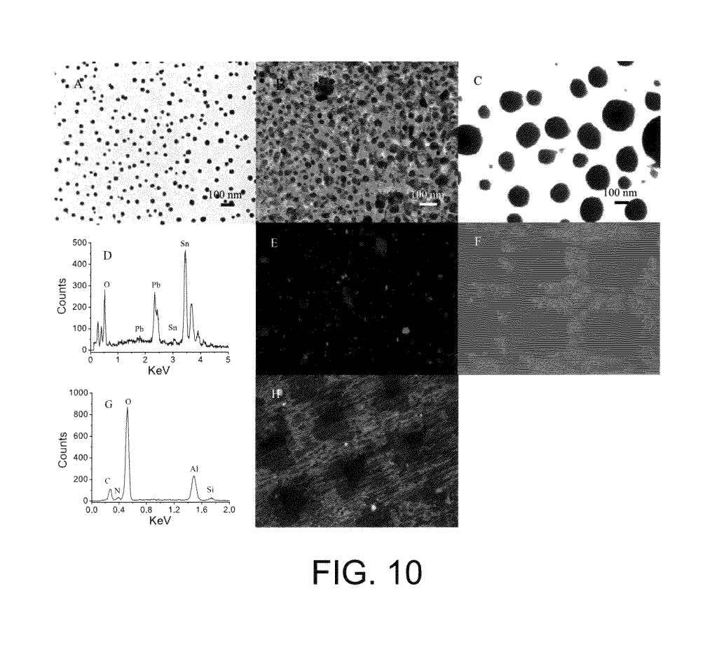

We describe a novel technique that depends on the solid-liquid phase transitions of nanoparticles to detect multiple proteins. In this thermally-addressed immunosorbent assay (TAISA), pure metal or alloy nanoparticles with a variety of compositions and melting temperatures are made by colloidal method and modified by antibodies; a solid surface (i.e., aluminum) is also modified by antibodies (Langmuir 2005, 21, 10907); the aluminum surface is immersed in a solution that contains the target antigen; after washing, the aluminum surface is immersed in a solution containing nanoparticle-labeled antibody (FIG. 10). In this sandwich detection configuration, the thermal property of immobilized nanoparticles is directly measured rather than that of antigen-antibody interaction. The signal transduction is based on a known but unexplored phenomenon: the temperature of a single component solid does not rise over its melting point until the entire solid is molten (Appl. Thermal Eng. 2003, 23, 251), thus the melting peak of the solid is sharp after a linear temperature rise process. A one-to-one correspondence can be created between the melting point of one type of nanoparticles and one type of protein. The melting temperature and the heat flow are used to distinguish and quantify the nature and concentration of the protein. Because the melting temperature of nanoparticles can be designed to be higher than those of the species contained in samples, the method is immune to colored or conductive species. In addition, the method can detect multiple proteins due to sharp melting peaks and multiple choices of phase change materials (i.e., metal, alloy, polymer and salt). In a typical thermal scan from 20 to 700° C., the number of different types of nanoparticles that can be detected in one run will reach 1000 if the peak width at half maximum is 0.6° C., meaning that the method has high level of multiplicity.

### EXPERIMENTAL SECTION

### Synthesis of Phase Change Nanoparticles

All chemicals used in this experiment are obtained from Sigma-Aldrich. The phase change nanoparticles of pure metals and alloys such as indium, tin, and lead-tin alloy nanoparticles are prepared by thermal decompositions of organometallic precursors (Chem. Mater. 2008, 20, 3656; J. Am. Chem. Soc. 2000, 122, 7114; Nano Lett. 2004, 4, 2047). The precursors are dissolved, or two types of precursors are mixed at stoichometric ratio and dissolved into ethylene glycol (EG) in the presence of polyvinyl-pyrrolidone (PVP). These precursors are decomposed at 200° C. under protection of nitrogen. After reacting for 30 min, the reactions are quenched by pouring the liquid into 200 ml of ethanol that is pre-cooled at 0° C. Then the nanoparticles are separated by using centrifugation, washed by ethanol for three time, and dried with nitrogen flow for future use.

Nanoparticle Characterizations.

A ZeissUltra 55 scanning electron microscope (SEM) working at accelerating voltage of 10 kV is used to image nanoparticles that are dispersed from suspension onto a conductive silicon surface. The compositions of nanoparticles are confirmed by an energy dispersive X-ray (EDX) detector. A JEOL 1011 transmission electron microscope (TEM) that is operated at 100 kV is used to image the nanoparticles. The melting temperatures and the fusion enthalpies of nanoparticles are measured by using a differential scanning calorimetry DSC (PerkinElmer DSC 7). 5 mg of nanoparticles is sealed inside an aluminum pan, and tested from 50 to 300° C. at a temperature ramp rate of 10° C./min. An empty aluminum pan is used as reference to determine the difference in heat flow of the sample and the reference. The DSC experiments provide heat fluxes in both melting and crystallization processes, but only melting curves are used in this work. The enthalpies of fusion of the nanoparticles are derived from peak areas using the data analysis software of DSC instrument. From the latent heats of fusion, the amount of phase change nanoparticles can be derived. The melting temperatures of indium, lead-tin, and tin nanoparticles are determined to be 156, 183, and 230° C., respectively, which are the same as the bulk counterparts because of the large diameters (J. Appl. Phys. 2005, 97, 034304; Chem. Phys. Lett. 2006, 429, 492; Phys. Rev. Lett. 2001, 87, 095505). Lead-tin alloy nanoparticles at the eutectic compositions have also been prepared by this method. FIGS. 10A-C show the TEM images of indium, lead-tin alloy, and tin nanoparticles. The crystallized structures of nanoparticles at room temperatures have been confirmed by selected area electron diffraction (SAED). Some of characterization results such as SEM images, SAED patterns, and DSC curves can be found in our previous papers (Appl. Phys. Lett. 2009, 95, 043701).

Surface Modification of Nanoparticles.

The surface modifications of nanoparticles are carried out as following. The lead-tin nanoparticles are dried and heated to 100° C. in an oven in atmosphere, which produces thin layers of oxide around nanoparticles. The existence of thin oxide layer has been verified by using energy dispersive X-ray analysis (EDX) (FIG. 10D). The oxide layer of nanoparticle is amine-modified by incubating in 10% (aminopropyl)triethoxysilane (APTES) in toluene for 1 hr at room temperature, which is followed by washing in toluene and in PBS solution. The surface modification of nanoparticles is confirmed by using fluorescent labeled bovine serum albumin proteins (BSA), which are covalently immobilized onto nanoparticles using a bifunctional cross-linker, disuccinimidyl suberate (DSS). FIG. 10E is a fluorescent image of BSA modified lead-tin nanoparticles. Furthermore, we have shown the surface oxidation of lead-tin thin films at the same heating condition, where the thin film is patterned to indicate fluorescent contrast. Lead-tin thin films are deposited onto silicon surfaces using electron beam evaporation. Then thin layers of photoresist are spun onto the films and exposed with UV light through a photomask that have patterns. After developing exposed portions, the films are treated in APTES vapor at 100° C., which is followed by removing unexposed photoresist. In the next, fluorescent BSA proteins are immobilized onto amine-modified surfaces by DSS. The micro-pattern on lead-tin film can be seen under fluorescent microscope, confirming the oxidization and modification of lead-tin film (FIG. 10F). Similar modifications have been done on thin films of tin and indium.

Surface Modification of Aluminum Surfaces.

In order to immobilize ligands on surface, the native oxide film on an aluminum surface is modified by putting the surface in a small vial that contains 0.1 ml of APTES. The vial is then heated in an oven at 100° C. for 1 hour. The APTES vapor will condense and react with oxide surface to form an amine-terminated monolayer. EDX spectrum confirms the oxidation and modification of aluminum (FIG. 10G). The surface modification has been further confirmed by immobilizing fluorescent BSA proteins on an aluminum surface with micropatterns. After washing the surface by DMSO, the amine-terminated surface is immersed in a DMSO solution containing DSS for 1 hr. The surface is washed for three times using DMSO and PBS, respectively, and incubated with fluorescent labeled BSA protein in PBS for 2 hrs. The un-reacted proteins are removed by washing with PBS: FIG. 10H shows the fluorescent micro-patterns on the aluminum surface.

Preparation of Cell Lysate.

The MDA-MB-231 human breast cancer cells are obtained from American Type Culture Collection (ATCC, Manassas, Va.). These cells are grown to confluence in T-75 tissue culture flasks using a protocol suggested by ATCC. Then the culture medium is removed and the cells are washed with a phosphate buffered saline (PBS at pH 7.4). The cells are lysed in 3 ml/flask of lysis buffer for 15 min at 4° C. with gentle rocking, where the lysis buffer is composed of 10 mM Tris-HCl at pH 7.6, 100 mM NaCl, 1 mM EDTA, and 1% Triton X-100. The detergent (Triton) is able to break cell membranes. The cell lysate is collected and centrifuged at 12,000 rpm for 10 minutes at 4° C. to remove debris. The supernatant of cell lysate is collected and stored in small aliquots at −20° C. until use. The total protein concentration of as-made cell lysate is determined to be 1.1 mg/ml using the DC Protein Assay reagent (Bio-Rad Laboratories, Hercules, Calif.) with bovine serum albumin as the standard.

### RESULTS AND DISCUSSION

### Thermal Detection of Avidin in Buffers

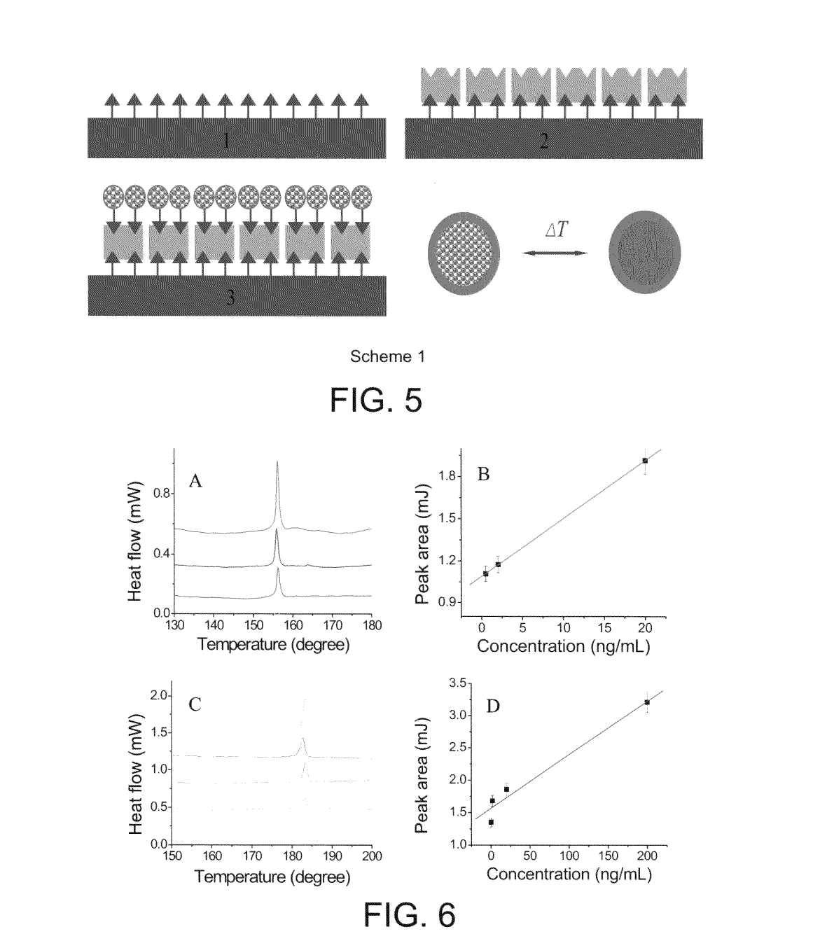

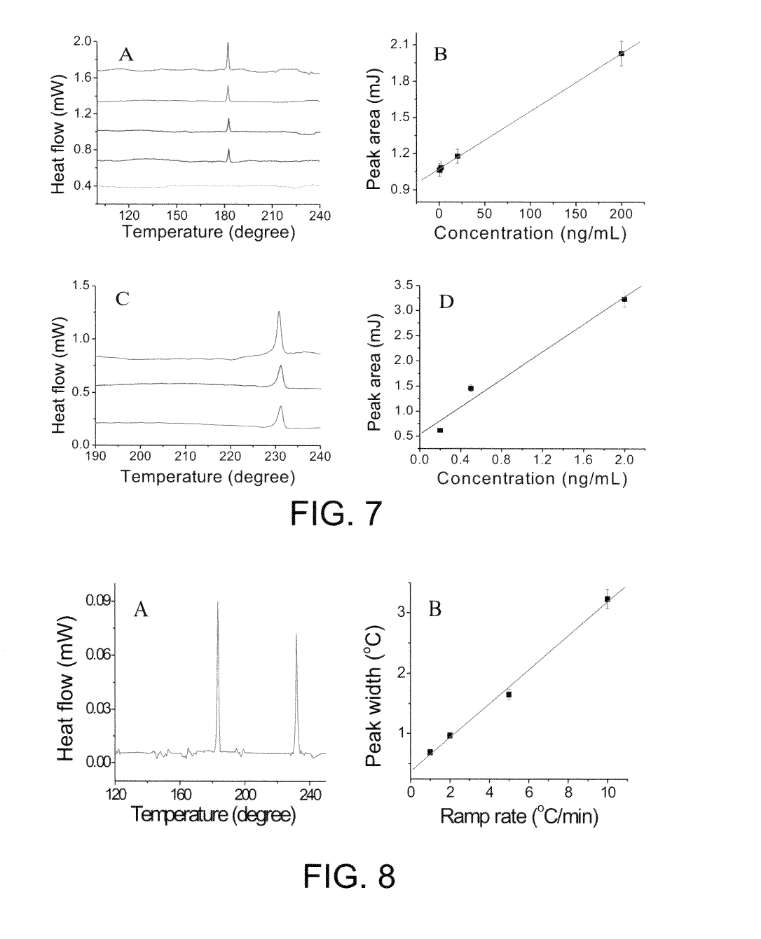

The thermally-address method is used to detect avidin in buffer by using phase change nanoparticles of indium and lead-tin alloy. The nanoparticles are modified to have amine groups. The aluminum surface with native oxide is also modified to have amine groups. The amine-modified nanoparticles and the aluminum surfaces are conjugated with biotins by incubating with amine reactive biotinylation reagent (NHS-LC-biotins) in anhydrous dimethyl sulfoxide (DMSO) for 1 hr at room temperature, which is followed by washing in DMSO and in phosphate buffered saline (PBS). The avidin detection is carried out by incubating biotin modified aluminum surface in solutions that contain avidin at certain concentrations for 1 hr. After rinsing, the surface is then incubated in biotin modified nanoparticles for 1 hr. Unconjugated nanoparticles are rinsed away, and immobilized ones are readout using DSC. FIG. 5A shows the DSC curves of indium nanoparticles immobilized on aluminum surfaces at different avidin concentrations, where the thermal scan is carried out from 50 to 300° C. at a ramp rate of 10° C./min. The single melting peaks at 156° C. confirm that indium nanoparticles have been immobilized onto aluminum surfaces. The measured heat flows decrease as the concentrations of avidin decrease (FIG. 5B). Similarly, we have also used lead-tin nanoparticles (atomic ratio of 63:37) to detect avidin in buffers. The avidin detection is carried out by incubating biotin modified aluminum surface in solutions containing different concentrations of avidin for 1 hr. After rinsing, the surface is incubated in biotin modified lead-tin nanoparticles for 1 hr. After rinsing the aluminum surface, the thermal signatures of lead-tin alloy nanoparticles are readout using DSC at a ramp rate of 10° C. per min. FIG. 5C shows the DSC curves of lead-tin alloy nanoparticles immobilized on aluminum surfaces at different avidin concentration, where nanoparticles melt at 183° C., and the heat flows decrease linearly as avidin concentration reduces (FIG. 5D).

### Thermal Immunoassay in Buffers.

The thermal immunoassay using phase change nanoparticles is carried out as following. The amine-modified nanoparticles and the aluminum surface are conjugated to anti-rabbit IgG by incubating them with disuccinimidyl suberate (DSS) in anhydrous DMSO in PBS (pH of 7.4) for 2 hr. The nanoparticles and the aluminum surface are then washed by PBS to remove excess DSS. The anti-rabbit IgG modified aluminum surface is incubated in solutions containing rabbit IgG at certain concentration for 1 hr. After rinsing, the surface is incubated in anti-rabbit IgG modified lead-tin nanoparticles for 1 hr. After rinsing the aluminum surfaces, the lead-tin nanoparticles are readout using DSC. FIG. 6A shows the melting peaks of lead-tin nanoparticles immobilized at different concentration of rabbit IgG. The cross-reactivity (selectivity) has been checked by human IgG (bottom curve), where 20 ng/ml human IgG does not lead to immobilization of nanoparticles. FIG. 6B shows the heat flow is proportional to the concentration of rabbit IgG.

In addition, human IgG in buffer is detected using tin nanoparticles in PBS (pH of 7.4). The surfaces of tin nanoparticles and aluminum surfaces are first modified with anti-human IgG. The anti-human IgG modified aluminum surface is incubated in solutions containing human IgG at certain concentration for 1 hr. After rinsing, the surface is incubated in anti-human IgG modified nanoparticles for 1 hr. FIG. 6C shows the melting peaks of tin nanoparticles immobilized on aluminum surfaces at several human IgG concentrations, where the nanoparticles melt at 230° C., and the heat flows decrease as the concentrations of human IgG reduce (FIG. 6D). Each DSC curve is flattened by using the commercial software of DSC instrument to remove the slope, which is induced by heat transfer difference between the sample cell and the reference cell.

### Multiplexed Thermal Immunoassay in Buffers.

The multiplicity of thermal detection is reflected in simultaneous detections of rabbit IgG and human IgG, where lead-tin nanoparticles and tin nanoparticles are modified with anti-IgGs of rabbit and human, respectively. In order to modify aluminum surfaces, both anti-IgGs of rabbit and human are mixed at the same molar ratio and immobilized on amine-ended aluminum surfaces. The multiplexed detection is done by incubating the modified aluminum surface in a mixture that contains 2 ng/ml rabbit IgG and 2 ng/ml human IgG in PBS (pH 7.4) for 1 hr. After rinsing the surface, it is incubated in a mixture of two types of modified nanoparticles for 1 hr. After rinsing for a second time, the aluminum surface is tested by DSC. FIG. 7A shows two melting peaks of tin and lead-tin nanoparticles at 183 and 230° C., respectively. The difference in the heat flows of the two peaks may be induced by differences in latent heats of indium and lead-tin alloy, sizes of two types of nanoparticles or grafting densities of anti-IgGs on them. The one-to-one correspondence created between protein and melting temperature allows the detections of multiple proteins in one experiment.

The multiplicity of the thermal method is dependent on the sharpness of individual peaks due to the issue of peak overlap. FIG. 7B shows the relation between the ramp rate and the peak width at half height of indium nanoparticles, where the peak width is proportional to the ramp rate, and the minimal peak width is 0.6° C. Considering the composition-dependent melting temperatures of alloys, nanoparticles with different melting temperature can be designed to have a large number of melting peaks based on phase diagrams. The sharp melting peaks, combined with the large number of different melting peaks of nanoparticles, enhance the multiplicity of detection by operating DSC at low ramp rate.

### Thermal Immunoassay in Cell Lysate.

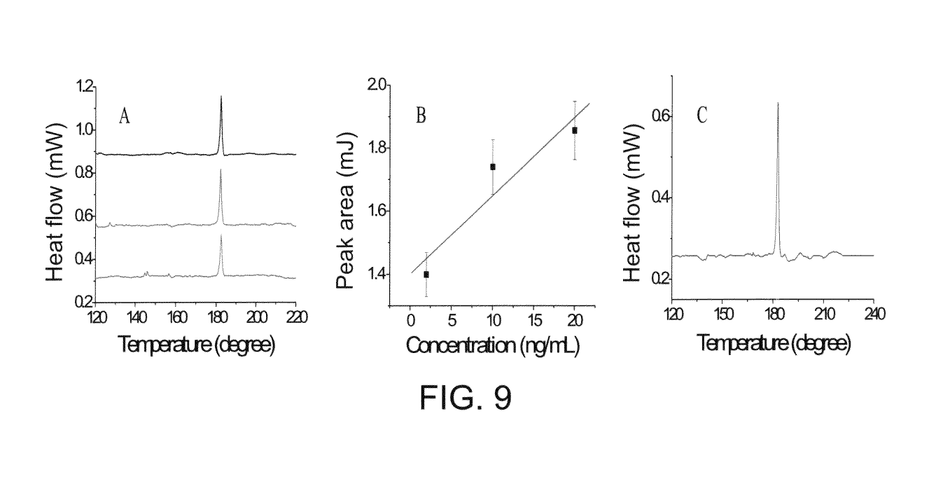

The thermal immunoassay has been carried out in complex cell lysate of human breast cancer cells. The cell lysate contains certain amount of human IgGs released from cell membranes. At first, rabbit IgGs are added at certain concentration in diluted cell lysates (total protein concentration is determined to be 1 μg/ml). The anti-rabbit IgG modified aluminum surfaces are incubated in solutions containing different concentration of rabbit IgG for 1 hr. After rinsing, the surface is incubated with anti-rabbit modified nanoparticles for 1 hr. After rinsing the surface again, the thermal signatures of lead-tin nanoparticles are readout using DSC at the ramp rate of 10° C. per min. FIG. 9A shows the DSC curves of lead-tin nanoparticles immobilized on aluminum surfaces at different rabbit-IgG concentration, where the heat flows decrease as the concentration of rabbit IgG decreases (FIG. 9B).

The concentration of human IgG in the diluted cell lysate (total protein concentration of 1 μg/ml) is determined by using anti-human IgG modified lead-tin alloy nanoparticles and aluminum surfaces. The surface modifications are carried out by using the same method as before. The anti-human IgG modified aluminum surface is incubated in the lysate for 1 hr. After rinsing, the surface is incubated with anti-human IgG modified lead-tin nanoparticles for 1 hr. Then the aluminum surfaces are taken out, washed using PBS to remove excess nanoparticles, and analyzed using DSC at the ramp rate of 10° C./min. FIG. 9C shows the melting curve of lead-tin nanoparticles immobilized on the aluminum surface. From the peak area, the concentration of human IgG in the dilute lysate (1 μg/ml total protein) is determined to be 182 ng/ml. As a rough comparison, 20% of proteins contained in normal human serum are IgG (Separation Purification Tech. 2009, 66, 242). If this value is valid for the human cancer cells in this study, the estimated concentration of human IgG (with total protein concentration of 1 μg/ml) would be 200 ng/ml, which is at the same order as the value we have derived using lead-tin nanoparticles.

In conclusion, we have shown the multiplexed detection of proteins using solid-liquid phase change nanoparticles as thermally addressed barcodes. A one-to-one correspondence is established between the type of nanoparticles and the type of proteins, thus multiple proteins can be detected simultaneously by using a combination of nanoparticles. The sensitivity can be further enhanced using nanoparticles with large latent heat of fusion, and reducing the grafting density of antibody on the nanoparticle. The melting peak and heat flow reflect the nature and concentration of protein, respectively. The melting temperature of nanoparticles can be designed to avoid interference from coexisting species in samples, thus bringing high sensitivity and multiplicity, and sample preparation benefits to the early detections of proteins. At last, the method is generic and can be used for the detections of disease markers in various body fluids.

### Example 4

### Brief Introduction

A big challenge for multiplexed detection of cancer biomarkers is that biomarker concentrations in body fluid differs several orders of magnitude. Existing techniques are not suitable to detect low and high concentration biomarkers (protein and DNA) at the same time, and liquid chromatography or electrophoresis is used to separate or purify target biomarkers before analysis. This Example describes a new broad-range biomarker assay using solid to liquid phase change nanoparticles, where a panel of metallic nanoparticles (i.e., metals and eutectic alloys) are modified with a panel of ligands to establish a one-to-one correspondence, and attached onto ligand-modified substrates by forming sandwiched complexes. The melting peak and fusion enthalpy of phase change nanoparticles during thermal analysis reflect the type and concentration of biomarkers, respectively. The thermal readout condition can be adjusted in such a way that multiple biomarkers with concentration difference over three orders of magnitude have been simultaneously detected under the same condition.

### Introduction

Although many cancer biomarkers have been discovered, it is clear that no single biomarkers can be used to detect lethal cancers from indolent ones (Science 2003, 299, 1679-1680; Nature Rev. Cancer 2007, 7, 309-315; Ann. Intern. Med. 1999, 131, 805-812; Proteomics 2005, 5, 4589-4596). The wide use of prostate specific antigen (PSA) screening has led to over-diagnosis and treatment of more than 1 million men in the United States. Parallel to effort of finding more specific biomarker, one feasible way of providing better predictive value is to detect multiple cancer biomarkers with low specificity simultaneously (Annu. Rev. Med. 2009, 60, 139-151; J. Natl. Cancer Inst 2003, 95, 661-668; Curr. Mol. Med. 2009, 9, 1017-1023; Science 2005, 307, 538-544; Nature Mater. 2005, 4, 435-446; Nature Biotechnol. 2003, 21, 47-51). However, a great challenge of existing techniques for multiple biomarker detection is that concentrations of cancer biomarkers differs several (eight to nine) orders of magnitude from pg/ml up to mg/ml, where a large proportion of biomarkers appear in low concentration range (Nature Rev. Cancer 2003, 3, 243-252; Nature Biotechnot 2008, 26, 1373-1378; Nature 2007, 450, 1235-1239). However, at conditions that low concentration ones can be detected, signals from high concentration ones are saturated for techniques such as DNA microarray, enzyme linked immune-sorbent assay (ELISA), polymerase chain reaction (PCR) and nanoparticle-based detection (Curr. Opin. Oncol. 2003, 15, 36-43; Urology 1999, 53, 228-235; Science 2000, 289, 1757-1760; Clin. Chem. 2002, 48, 1178-1185; Anal. Chem. 2004, 76, 5414-5417; Anal. Chem. 2008, 80, 2805-2810; Science 1998, 281, 2016-2018). Briefly, DNA microarray has identical feature, binding capacity, and narrow range of detection concentration (Nucl. Acids. Res. 2007, 35, 4154-4163; Proc. Natl. Acad. Sci. 2002, 99, 7554-7559; PLoS ONE. 2009, 4, e7088). The signal-target ratio in ELISA depends on conjugation of fluorescence label on each antibody, and is constant after surface modification, and cannot adjust concentration range of detection (Anal. Chim. Acta. 1999; 378, 219-224; Anal. Biochem. 2005, 347, 159-161). PCR provides saturated signals at high biomarker or enzyme concentration (Anal. Chem. 2006, 78, 7997-8003; Am. J. Clin. Pathol. 2001, 115, 439-447). Most of these techniques do not have high multiplicity; and biomarkers of different types such as DNAs and proteins cannot be detected simultaneously at the same condition. As a result liquid chromatography or electrophoresis has to be used to separate biomarkers prior to analysis.

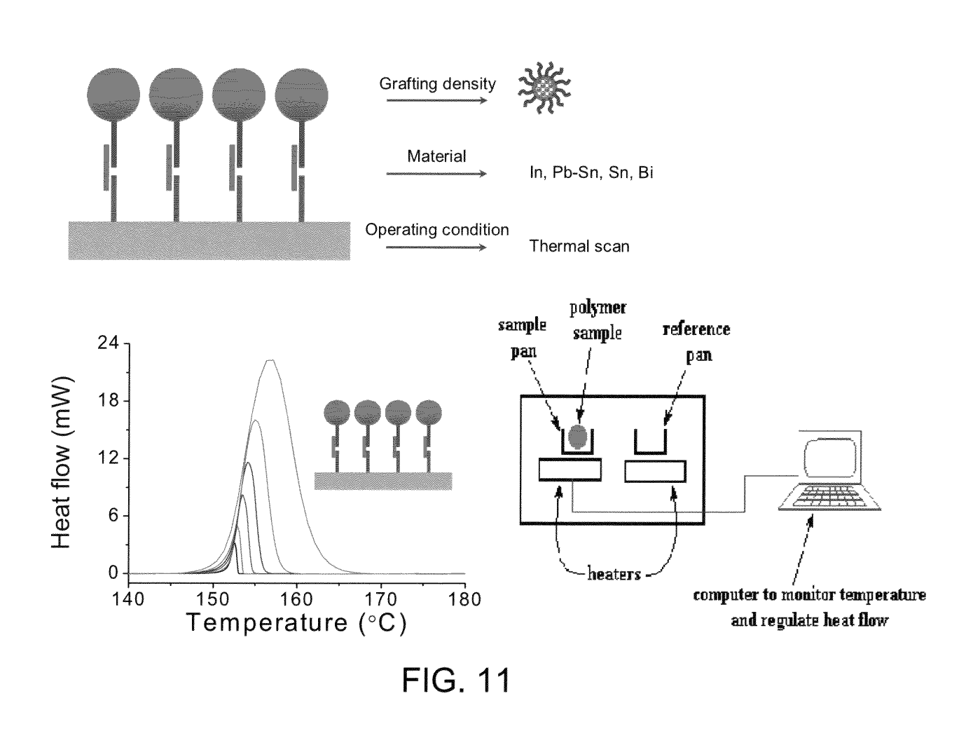

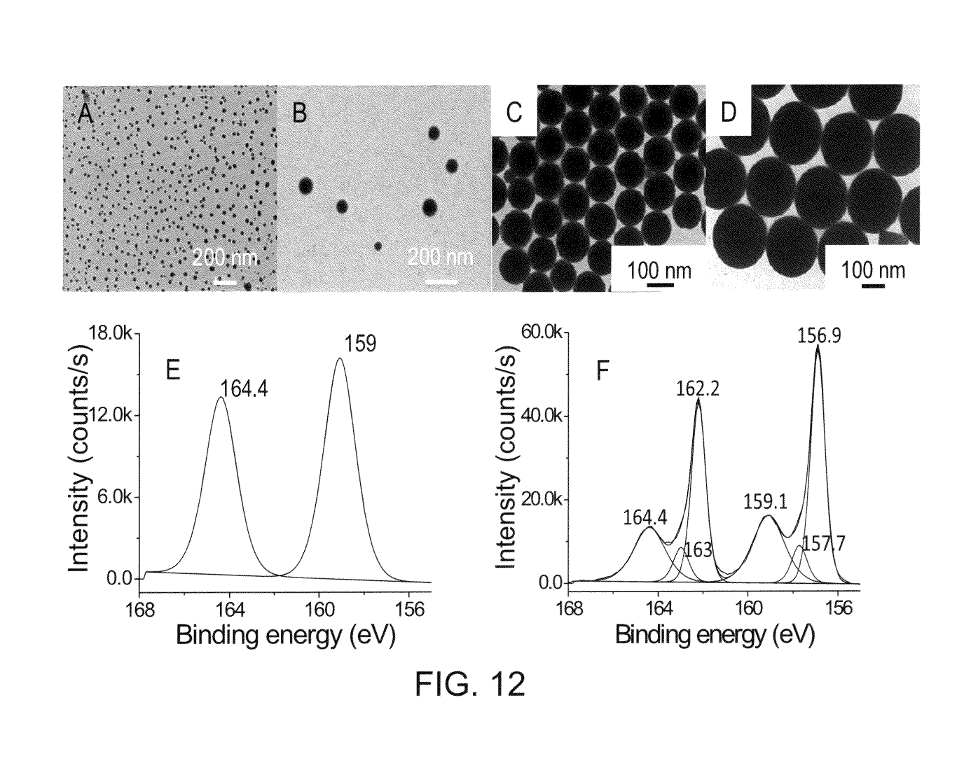

This Example describes a new biosensing method based on solid-liquid phase change nanoparticles, where each type of nanoparticles is conjugated with ligands of according protein and DNA biomarkers, and immobilized on ligand-modified substrates by forming double helix DNA chain or sandwiched antibody-antigen complex. The type and concentration of biomarkers are reflected in melting temperature and fusion enthalpy of nanoparticles in differential scanning calorimetry (DSC). The characters of nanoparticles such as nanoparticle size, materials, grafting density, and readout condition (i.e., thermal ramp rate) can be adjusted so that multiple biomarkers with concentration difference of over three orders of magnitude can be detected at the same time and under the same condition (FIG. 11). It is anticipated that concentration range can be extended to 6-10 orders of magnitude by optimizing detection conditions.

### Materials and Method

### Materials and Chemicals

Oligonucleotides are customer-synthesized at Integrated DNA Technologies, Inc (Coralville, Iowa) with desired sequences. Capture single stranded DNA: ssDNA1 (5′-/5AmMC6/ATTATTATTATGTGGTTGCTGTGT-3′) (SEQ ID NO: 1), ssDNA2 (5′-5AmMC6/ATTATTAAGCGTGTACTGAACT-3′) SEQ ID NO: 2; target ssDNA1 (3′-TACACCAACGACACAA ATGTTATTAGG-5′) SEQ ID NO: 3, ssDNA2 (3′-TCGCACATGACTTGAAATGTTATTAGG-5′) SEQ ID NO: 4; probe ssDNA1 (5′-TTAC AATAATCCATTATTATTA/3ThioMC3-D-3′) SEQ ID NO: 5, ssDNA2 (5′-TTACAATAATCCATTATTATTA/3Thio-MC3-D-3′) SEQ ID NO: 6. Human IgG and its antibody, and prostate specific antigen (PSA) are obtained from Sigma. Two types of mouse anti-human PSA monoclonal antibodies (CHYH1 and CHYH2) are obtained from Anogen/Yes Biotech Laboratory. Organometallic precursors such as indium acetate, lead acetate, bismuth acetate, tin acetate, and polyvinylpyrrolidone (PVP, molecular weight of 11,000) and ethylene glycol are from Aldrich. Disuccinimidyl suberate (DSS), N-succinimidyl(4-iodoacetyl)aminobenzoate (SIAB) as well as tris(2-carboxyethyl) phosphine (TCEP) are obtained from Thermo Fisher Scientific (Rockford, Ill.). Anhydrous dimethyl sulfoxide (DMSO), n-propyltriethoxysilane and 3-aminopropyltriethoxysilane (APTES) are obtained from VWR (West Chester, Pa.). Phosphate buffer saline, bovine serum albumin (BSA) and Tween-20 are obtained from Sigma. All immuno-reagents are dissolved in pH 7.4 phosphate saline (PBS) buffer (0.01 M in phosphate, 0.14 M NaCl, 2.7 mM KCl) unless otherwise noted. Ultrapure water is used throughout experiment.

### Nanoparticle Synthesis and Characterization

The phase change nanoparticles of metals and alloys have been synthesized by thermal decompositions of organometallic precursors (indium acetate, lead acetate, tin acetate, or bismuth acetate) with stoichiometric ratios (Appl. Phys. Lett. 2009, 95, 233101). In brief, the precursors and polyvinylpyrrolidone (molecular weight of 11000) are dissolved in ethylene glycol and stirred for 10 minutes to make a homogeneous solution. After the mixture is refluxed at 200° C. for 20 minutes to decompose precursors under nitrogen protection, the product is quenched by pouring the mixture in ethanol pre-cooled at 0° C. The as-prepared nanoparticles are then centrifuged at 3000 rpm for 10 minutes. After removing supernatant, the nanoparticles are washed by ethanol for three times. A droplet of nanoparticles in ethanol solution is deposited onto a carbon grid. After solvent evaporation, a JEOL 1011 transmission electron microscope (TEM) operated at 100 kV is used to determine the size and shape of nanoparticles. A Zeiss Ultra 55 scanning electron microscope (SEM) at accelerating voltage of 10 kV is used to derive morphology and size distribution of nanoparticles. The compositions of nanoparticles are analyzed by using an energy dispersive X-ray (EDX) detector. A differential scanning calorimetry (PerkinElmer DSC7) is used to study thermophysical behaviors of nanoparticles. An Olympus BX51 M fluorescence microscopy is used to measure the fluorescence of fluorescein isothiocyanate labeled bovine serum albumin (FITC-BSA) immobilized on thin films.

### Surface Modification

The surface oxidized nanoparticles are modified with amine-functionalized monolayers by reacting with 3-aminopropyltriethoxysilane (APTES). In order to control ligand grafting density, a mono-functional silane (i.e., n-propyltriethoxysilane) is used to co-modify nanoparticles at a variety of molar ratios (1:0, 1:1, 1:2, 1:5, and 1:10). The modification is performed by immersing nanoparticles in toluene that contain 5% (v/v) of the mixture of silane at certain ratio. After reacting for 1 hr at room temperature, nanoparticles are washed with toluene and dimethyl sulfoxide (DMSO) for three times to remove excess silane. To conjugate single stranded DNA (ssDNA) onto nanoparticles, the amine groups at nanoparticles are activated with 1 mM N-succinimidyl(4-iodoacetyl)aminobenzoate (SIAB) in DMSO for 1 hr; disulfide-containing probe ssDNA is reduced with 0.25M tris(2-carboxyethyl)phosphine (TCEP) at pH 4.5 in tris(hydroxymethyl)aminomethane (TE) buffer at 37° C. for 3 hr; after removing excess SIAB by centrifugation, nanoparticles are incubated with 20 μM 3′ thiolated probe ssDNA in a phosphate buffer (PBS, pH 8.0) at room temperature. In order to conjugate protein, amine-modified nanoparticles are activated by incubating with 1 mM DSS (disuccinimidyl suberate) crosslinker dissolved in DMSO for 1 hr, which is followed by incubating with antibody in PBS for 3 hr. In order to immobilize ligands on aluminum pan for DSC readout, the native oxide on aluminum is modified to have amine functional groups by vapor deposition of APTES at 100° C. for 1 hr. The surface is washed by DMSO and then immersed in DMSO containing DSS (1 mM) for 1 hr. After washing with DMSO and PBS for three times, the aluminum surface is incubated with amine-functionalized capture ssDNA in PBS, or a mixture of capture ssDNA and antibodies in 4× saline sodium citrate (SSC) buffer for 2 hr. Unreacted ssDNAs or proteins are removed by washing thoroughly with buffer solution that contains 1% (w/v) BSA and 0.05% Tween-20.

### Biomarker Detection

Antibody or ssDNA modified aluminum pans are incubated with target ssDNA or protein (antigen) at various concentrations inside a buffer solution at room temperature for 3 hr. After washing with PBS or SSC buffer, the aluminum pan with immobilized ssDNA or proteins is immersed in buffer (PBS or SSC) that contains ssDNA or antibody modified nanoparticles for 1 hr. The substrate is rinsed thoroughly with buffer and dried for thermal readout. A PerkinElmer DSC (DSC7) is used to determine the melting point and fusion enthalpy of immobilized nanoparticles at certain ramp rate, where an empty aluminum pan is used as reference to derive the difference in heat flow of sample and reference. The nature and concentration of biomarkers can be derived from the melting point and heat flow of nanoparticles based on one-to-one correspondence between nanoparticles and biomarker. The DSC baselines are flattened using Origin software to remove slope and noise after collection.

### Results and Discussion

The multiplicity of thermal detection depends on peak width, thermal scan range and type of nanoparticles. In the temperature range of 100-1000° C., although the available number of metals is limited, the number of eutectic alloys with sharp melting peak is large, and can be identified from phase diagram. The peak width depends on heat transfer rate, thermal conductivity of atmosphere, and thermal ramp rate, and could be derived from Gray's model based on energy conservation and Newton's law (Thermochim. Acta. 1994, 231, 203-213). The melting peak shape for a small amount of sample has been shown to consist of two half-peaks: the first half-peak has straight line slope, and the second half-peak shows an exponential decay. The width of melting peak can be derived from a modified equation

$$w = 0.5 β \cdot Δ t = 0.5 β \cdot ( {t}_{1} + {t}_{2} ) = 0.5 β \cdot [ {RC}_{s} ( \sqrt{1 + \frac{2 Δ H}{{RC}_{S}^{2} β}} - 1 ) + {RC}_{s} ln ( 100 ) ] \qquad ( 1 )$$

where R is thermal resistance, Cₛ is the total sample heat capacity, ΔH is the latent heat of fusion, β is heating rate; t₁ and t₂ are time needed for solid to melt, and time needed for temperature to catch up with programmed temperature, respectively. Theoretically, t₂ is infinitely long, but for practical purpose, t₂ is defined as the time for heat flow fall below 1% of its maximum. The sensitivity and the concentration range of detection can be adjusted by changing materials, particle size and operating condition (ramp rate). Assuming that nanoparticles have uniform radius (r), and the grafting density of ligand on nanoparticle is α, the number of biomarkers (n) that can be detected will be

$$n = \frac{3 Q α}{4 π {r}^{3} ρ \cdot β \cdot Δ H} \qquad ( 2 )$$

where Q is detectable heat flow (determined by instrument), and p is density of nanoparticle.

Indium, lead-tin alloy, tin and bismuth nanoparticles (melting temperatures of 156, 183, 231, and 271° C.) have uniform size at 30, 100, 100, and 200 nm (FIG. 12A-12D), respectively. The as-made nanoparticles are surface-oxidized by heating in air at 70° C. for 30 min. The formation of oxide shell around metallic nanoparticles is confirmed by XPS. Two asymmetrical peaks observed at 159 and 164.4 ev, as shown in FIG. 12E, correspond to Bi 4f_{7/2} and Bi 4f_{5/2} of bismuth oxide, which reflect formation of oxide at nanoparticles. After sputtering nanoparticles with argon ion for 10 seconds at 3 kV and 14 μA, two peaks which are 4f_{7/2} and 4f_{5/2} peaks of bismuth metal appear at 156.9 and 162.2 eV (FIG. 12F) (J. Mol. Catal. A: Chem. 2007, 261, 167-171; J. Appl. Phys. 1989, 66, 2045-2048). Similar oxide core-metal shell structures have also been found in other three types of nanoparticles.

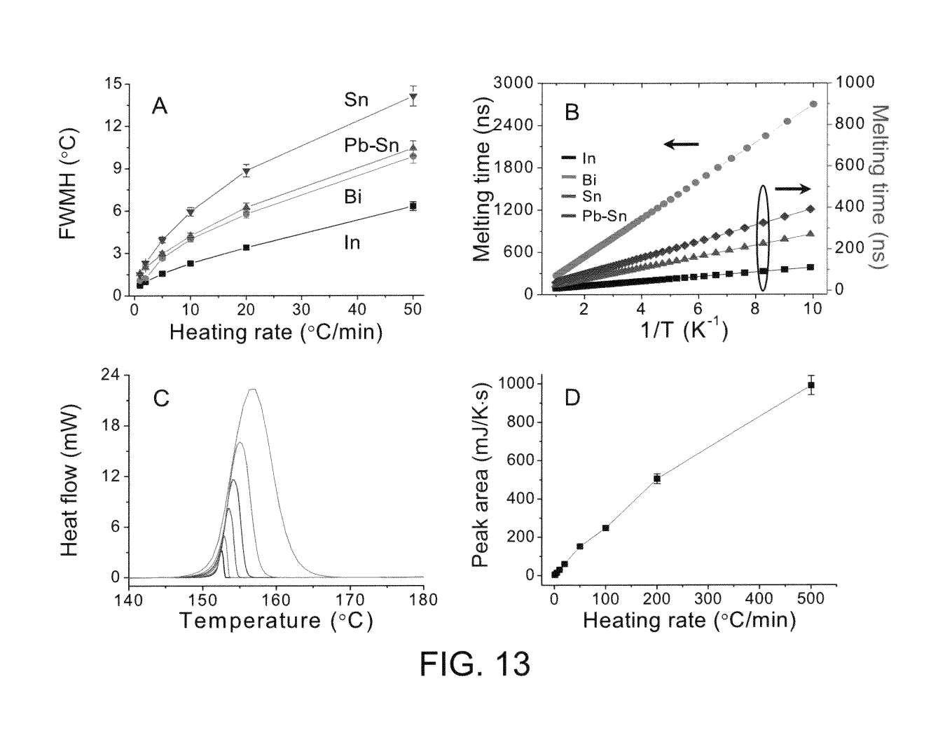

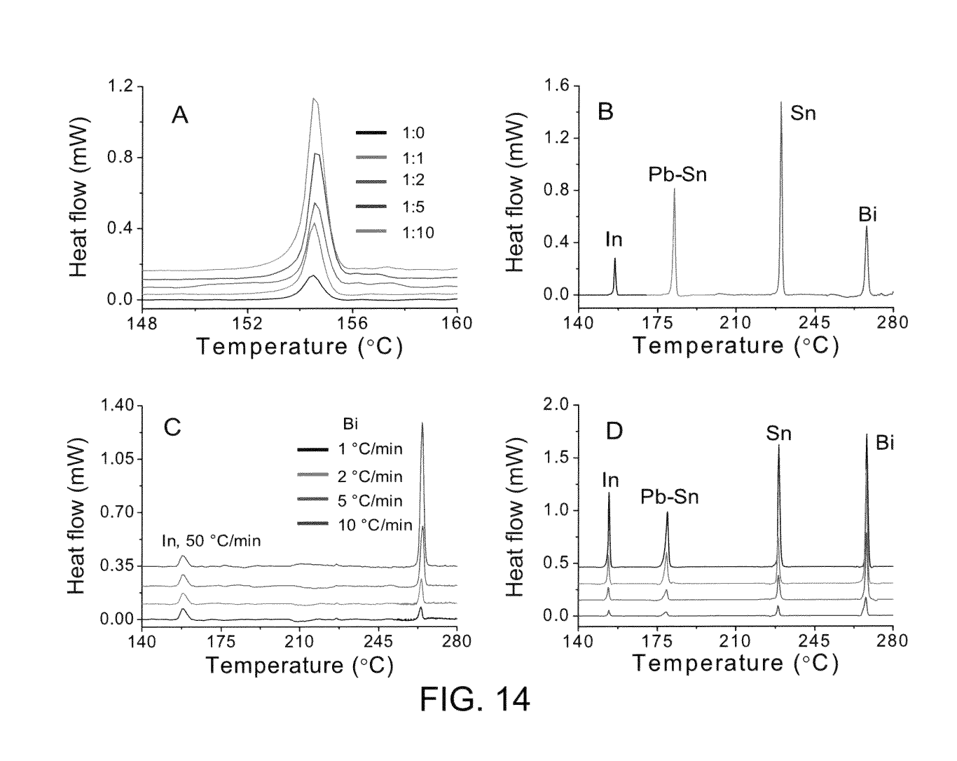

FIG. 13A shows ramp rate dependent peak width for different nanoparticles, where half widths at maximum height (HWMH) of nanoparticles increase as ramp rate increases from 1 to 50° C./min. The existence of melting peaks confirms that metallic nanoparticles preserve in oxide shells. In the case of tin nanoparticles, the peak width changes from 1.5 to 14° C. when ramp rate increases from 1 to 50° C./min. By fitting data with equation (1), the thermal resistance and total sample heat capacity are determined to be 5.0×10⁻³° C./mW and 33.8 mJ/° C., respectively. On the other hand, the contribution of nanoparticle on melting time can be derived from

$$\frac{( {T}_{s} - {T}_{m} ) \cdot τ}{( \frac{{ρ}_{l} {Q}_{l}}{{k}_{l}} )} = {r}_{p}^{2} [ \frac{1}{3} {( \frac{r}{{r}_{p}} )}^{3} - \frac{1}{2} {( \frac{r}{{r}_{p}} )}^{2} + \frac{1}{6} ] \qquad ( 3 )$$

where Tₛ and Tₘ are surface temperature and melting point of nanoparticles, respectively; τ is the melting time when the radius of solid is r, rₚ is radius of nanoparticles; Qₗ is the latent heat of fusion of nanoparticles; kₗ is thermal conductivity of nanoparticles; and ρₗ is density of nanoparticles. kₗₙ is 81.8 W·m⁻¹·K⁻¹, rₗₙ is 50 nm, Qₗₙ is 28.52 kJ/kg, and ρₗₙ is 7310 kg/m³. Replace all symbols with number and let r equal to 0 nm, equation (3) is rewritten as τ (Tₛ−Tₘ)=1.1×10⁻¹·K⁻¹. Assuming Tₛ-Tₘ=0.1 K, τ is 11 ns. A linear relation exists between the melting time and 1/(Tₛ−Tₘ) as shown in FIG. 13B for four types of nanoparticles. The difference in peak width induced by melting time difference is less than 0.01° C. The small contribution on peak width suggests that heat transfer inside nanoparticles is not important on peak width; instead, the heat transfer from heater through air to aluminum pan and nanoparticles is important. FIG. 13C shows melting curves of indium nanoparticles at ramp rates of 1, 2, 5, 10, 20, and 50° C./min. The similarity among peaks suggests surface oxidation is not incremental after reaching equilibrium. The peak shifts to high temperature as ramp rate increases. The peak area is linearly proportional to heating rate in the range from 1 to 500° C./min (FIG. 13D). Providing the equipment sensitivity depends on RMS (root-mean-square) noise, the large heat flow means that detectable limited can be lowered by 500 times.

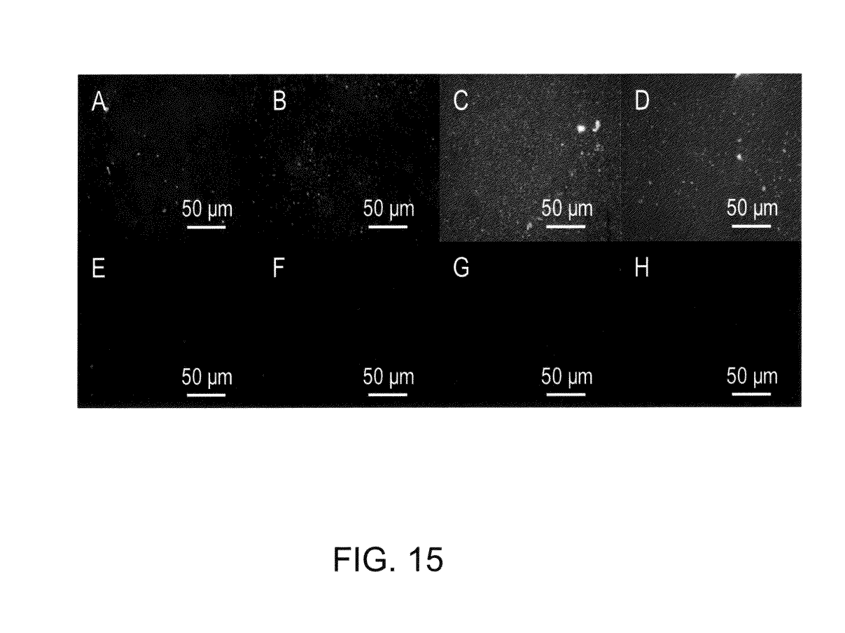

The ligand grafting density around nanoparticles affects the detection range and sensitivity. The grafting density of silane on each type of material is first derived from fluorescence intensity after cross-linking fluorescence labeled bovine serum albumin at amine terminated thin films evaporated onto silicon substrates. The different metallic/alloy thin films with a thickness of 100 nm are produced on the silicon substrates by thermally deposited corresponding metallic/alloy powder. From relative intensities of fluorescence images (FIG. 15), the ratio of ligand grafting densities of silane on indium, lead-tin alloy, tin, and bismuth thin films are determined as 1:1:2:2. FIG. 14A shows grafting density dependent melting of immobilized nanoparticles, where melting curves from top to bottom correspond to 10 nM ssDNA1, respectively. Changing molar ratio from 1:0 to 1:10 leads to larger melting peaks, which is consistent with equation 2 where the peak area is larger at lower grafting density providing others are the same. Heat flow can be changed using materials with different latent heats. The latent heats for indium, lead, tin, and bismuth are 28.5, 23, 58.5, and 52 J/g respectively, and that for lead-tin alloy (Pb₃₇Sn₆₃) is 45.4 J/g which is derived by taking mass fraction (x) into consideration using H=xHₐ+(1−x)H_{b}, and Hₐ and H_{b} are latent heats of metals. FIG. 14B shows DSC curves of four types of nanoparticles immobilized on aluminum plates after detecting 10 nM target ssDNA1. The peak area of immobilized tin nanoparticles is the largest because the latent heat of tin (58.5 J/g) is larger than other type nanoparticles.

The thermal scan can be programmed with multiple ramp rates. FIG. 14C shows four DSC curves of indium and bismuth nanoparticles, where melting peaks of indium nanoparticles are collected after detecting 100 μM ssDNA1 at 50° C./min; the melting peaks of bismuth nanoparticles are collected after detecting 100 nM ssDNA2 at variable ramp rates from 1 to 10° C./min. A discontinuity in heat flow induced by ramp rate change at 200° C. is removed for display purpose. The variable rate scan is important for several reasons: (1) overlapped peaks can be separated from each other; (2) peak area (heat flow and sensitivity) can be varied; and (3) analysis time can be reduced by quickly scanning between peaks. These nanoparticles are used to detect four types of biomarkers (two types of DNA and two types of protein) in the same SSC buffer. FIG. 14D shows four DSC curves, where each of curve contains four types of nanoparticles immobilized on aluminum. Indium nanoparticles are used to detect ssDNA1 (100 nM to 100 pM); lead-tin nanoparticles are used to detect ssDNA2 (100 nM to 100 pM); tin nanoparticles are used to detect PSA (100 to 5 pg/ml); and bismuth nanoparticles are used to detect human IgG (10 to 0.5 ng/ml). The concentration detection range can be estimated using following parameters. DSC allows ramp rate to be adjusted in the range of 0.02 to 500° C./min, meaning that the detectable heat flow (concentration range) can be adjusted over four orders of magnitude. The diameter of nanoparticles can be changed from 30 to 150 nm; the grafting sites on nanoparticle can be any number from one to 4π·1000 (ACS Nano 2009, 3, 418-424); the latent heat of fusion can be changed from 23 J/g for indium to 205 J/g for copper. According to equation (2), the upper limit of concentration will be 2×10¹³ and the lower limit of concentration will be 9.2×10², thus concentration range is derived as ten orders of magnitude. This method has been used successfully in detecting two DNA biomarkers whose concentrations are at 100 nM and 100 pM, spanning three orders of magnitude (FIG. 14C).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim at least the following:

1. A probe, comprising:
a nanoparticle having an outer structure and an inner area, wherein the outer structure encapsulates the inner area, wherein a phase change material is within the inner area, wherein the melting point of the outer structure is greater than the melting point of the phase change material, wherein a first targeting moiety is attached to the surface of the outer structure.

2. The probe of claim 1, wherein the phase change material is a solid-to-liquid phase change material.

3. The probe of claim 2, wherein the solid-to-liquid phase change material is selected from the group consisting of: indium, tin, lead, bismuth, gold, silver, a salt, paraffin wax, and a combination thereof.

4. The probe of claim 2, wherein the phase change material is a reversible solid-to-liquid phase change material.

5. The probe of claim 1, wherein the nanoparticle has a diameter of about 10 nm to 1000 nm.

6. The probe of claim 1, wherein the targeting moiety is selected from the group consisting of: a polypeptide, an antibody, an antigens, a nucleic acid, a polysaccharide, a sugars, a fatty acid, a steroids, a purine, a pyrimidine, a ligand, and a combination thereof.

7. The probe of claim 1, wherein the melting point of the phase change material is about 100 to 700° C.

8. The probe of claim 1, wherein the outer structure is made of a material selected from the group consisting of: silica, alumina, titania, polymer, an oxide of the phase change material, and a combination thereof.

9. The probe of claim 1, wherein the outer structure has a thickness of about 2 to 200 nm.

10. The probe of claim 1, wherein the phase change material is selected from the group consisting of: a solid-to-solid, a solid-to-liquid, a solid-to-gas, and a liquid-to-gas, phase change material.

11. A probe complex, comprising:
a probe, a capture moiety, and a target,
wherein the probe is a nanoparticle having an outer structure and an inner area, wherein the outer structure encapsulates the inner area, wherein a phase change material is within the inner area, wherein the melting point of the outer structure is greater than the melting point of the phase change material, wherein a targeting moiety is attached to the surface of the outer structure,
wherein a second targeting moiety is attached to the capture moiety,
wherein the targeting moiety has an affinity for a first portion of the target, wherein the second targeting moiety has an affinity for a second portion of the target, wherein the first portion of the target and the second portion of the target are not the same.

12. The probe of claim 11, wherein the phase change material is selected from the group consisting of: a solid-to-solid, a solid-to-liquid, a solid-to-gas, and a liquid-to-gas, phase change material.

13. The probe of claim 12, wherein the solid-to-liquid phase change material is selected from the group consisting of: indium, tin, lead, bismuth, gold, silver, a salt, paraffin wax, and a combination thereof.

14. The probe of claim 11, wherein the capture moiety is a magnetic capture moiety, wherein the second targeting moiety is attached to the magnetic capture moiety, wherein the probe complex includes the second targeting moiety and the magnetic capture moiety.

15. The probe of claim 11, wherein the capture moiety is a substrate, wherein the second targeting moiety is attached to the substrate.

16. The probe of claim 15, wherein the substrate is selected from the group consisting of: an aluminum substrate, a gold substrate, a ceramic substrate, a glass substrate, a silicon dioxide substrate, and a silicon substrate.

17. A particle comprising:
a plurality of nanoparticles enclosed by a particle shell structure, wherein each nanoparticle has an outer structure and an inner area, wherein the outer structure encapsulates the inner area, wherein a phase change material is within the inner area, wherein the melting point of the outer structure is greater than the melting point of the phase change material.

18. The particle of claim 17, wherein the plurality of nanoparticles is about 2 to 50.

19. The particle of claim 17, wherein the plurality of nanoparticles includes 2 to 50 different types of nanoparticles, wherein each type of nanoparticle includes a different type of phase change material, wherein each different type of phase change material has a different melting temperature than each of the other types of nanoparticles.

20. The particle of claim 17, wherein the phase change material is selected from the group consisting of: a solid-to-solid, a solid-to-liquid, a solid-to-gas, and a liquid-to-gas, phase change material.

21. The particle of claim 17, wherein each nanoparticle has a diameter of about 10 nm to 1000 nm.

22. The particle of claim 17, wherein the particle shell structure has a thickness of about 1 nm to 100 nm.

23. The particle of claim 17, wherein the particle shell structure is made of a material selected from a group consisting of: silica, alumina, titania, polymer, an oxide of the phase change material, and a combination thereof.

24. A method of detecting a target, comprising:
mixing a probe, a capture moiety, and a target,
wherein the probe is a nanoparticle having an outer structure and an inner area, wherein the outer structure encapsulates the inner area, wherein a phase change material is within the inner area, wherein the melting point of the outer structure is greater than the melting point of the phase change material, wherein a first targeting moiety is attached to the surface of the outer structure,
wherein a second targeting moiety is attached to the capture moiety,
wherein the first targeting moiety has an affinity for a first portion of the target, wherein the second targeting moiety has an affinity for a second portion of the target, wherein the first portion of the target and the second portion of the target are not the same;
forming a probe complex including the probe, the second targeting moiety, and the target, wherein the first targeting moiety is attached to the first portion of the target, wherein the second targeting moiety is attached to the second portion of the target;
separating the probe complex from the other components of the mixture;
heating the probe complex; and
determining the melting point of the phase change material, wherein the melting point is used to identify the phase change material, wherein identifying the phase change material is used to determine the target.

25. The method of claim 24, wherein the capture moiety is a magnetic capture moiety, wherein the second targeting moiety is attached to the magnetic capture moiety, wherein the probe complex includes the second targeting moiety and the magnetic capture moiety.

26. The method of claim 24, wherein the capture moiety is a substrate, wherein the second targeting moiety is attached to the substrate.

27. The method of claim 26, wherein the substrate is a substrate selected from the group consisting of: an aluminum substrate, a gold substrate, a ceramic substrate, a glass substrate, a silicon dioxide substrate, and a silicon substrate.

28. A method of detecting a target, comprising:
mixing a first probe, a second probe, a first capture moiety, a first target, and a second target,
wherein the first probe is a nanoparticle having an outer structure and an inner area, wherein the outer structure encapsulates the inner area, wherein a first phase change material is within the inner area, wherein the melting point of the outer structure is greater than the melting point of the first phase change material, wherein a first targeting moiety is attached to the surface of the outer structure,
wherein the second probe is a nanoparticle having an outer structure and an inner area, wherein the outer structure encapsulates the inner area, wherein a second phase change material is within the inner area, wherein the melting point of the outer structure is greater than the melting point of the second phase change material, wherein the melting point of the first phase change material is different than the melting point of the second phase change material, wherein a second targeting moiety is attached to the surface of the outer structure,
wherein a third targeting moiety is attached to the first capture moiety,
wherein the first targeting moiety has an affinity for a first portion of the first target, wherein the third targeting moiety has an affinity for a second portion of the first target, wherein the first portion of the first target and the second portion of the first target are not the same,
wherein the second targeting moiety has an affinity for a first portion of the second target, wherein the third targeting moiety has an affinity for a second portion of the second target, wherein the first portion of the second target and the second portion of the second target are not the same;
forming a first probe complex including the first probe, the third targeting moiety, and the first target, wherein first targeting moiety is attached to the first portion of the first target, wherein the third targeting moiety is attached to the second portion of the first target;
forming a second probe complex including the second probe, the third targeting moiety, and the second target, wherein the second targeting moiety is attached to the first portion of the second target, wherein the third targeting moiety is attached to the second portion of the second target;
separating the first probe complex and the second probe complex from the other components of the mixture;
heating the first probe complex and the second probe complex;
determining a first melting point of the first phase change material, wherein the first melting point is used to identify the first phase change material, wherein identifying the first phase change material is used to determine the first target; and
determining a second melting point of the second phase change material, wherein the second melting point is used to identify the second phase change material, wherein identifying the second phase change material is used to determine the second target.

29. A method of detecting a target, comprising:
mixing a first probe, a second probe, a first capture moiety, a second capture moiety, a first target, and a second target,
wherein the first probe is a nanoparticle having an outer structure and an inner area, wherein the outer structure encapsulates the inner area, wherein a first phase change material is within the inner area, wherein the melting point of the outer structure is greater than the melting point of the first phase change material, wherein a first targeting moiety is attached to the surface of the outer structure,
wherein the second probe is a nanoparticle having an outer structure and an inner area, wherein the outer structure encapsulates the inner area, wherein a second phase change material is within the inner area, wherein the melting point of the outer structure is greater than the melting point of the second phase change material, wherein the melting point of the first phase change material is different than the melting point of the second phase change material, wherein a second targeting moiety is attached to the surface of the outer structure,
wherein a third targeting moiety is attached to the first capture moiety,
wherein a fourth targeting moiety is attached to the second capture moiety,
wherein the first targeting moiety has an affinity for a first portion of the first target, wherein the third targeting moiety has an affinity for a second portion of the first target, wherein the first portion of the first target and the second portion of the first target are not the same,
wherein the second targeting moiety has an affinity for a first portion of the second target, wherein the fourth targeting moiety has an affinity for a second portion of the second target, wherein the first portion of the second target and the second portion of the second target are not the same;
forming a first probe complex including the first probe, the third targeting moiety, and the first target, wherein first targeting moiety is attached to the first portion of the first target, wherein the third targeting moiety is attached to the Second portion of the first target;
forming a second probe complex including the second probe, the fourth targeting moiety, and the second target, wherein the second targeting moiety is attached to the first portion of the second target, wherein the fourth targeting moiety is attached to the second portion of the second target;
separating the first probe complex and the second probe complex from the other components of the mixture;
heating the first probe complex and the second probe complex;
determining a first melting point of the first phase change material, wherein the first melting point is used to identify the first phase change material, wherein identifying the first phase change material is used to determine the first target; and
determining a second melting point of the second phase change material, wherein the second melting point is used to identify the second phase change material, wherein identifying the second phase change material is used to determine the second target.

30. A method of detecting a target, comprising:
mixing a probe, a capture moiety, and a target,
wherein the probe is a nanoparticle made of a phase change material, wherein a first targeting moiety is attached to the surface of the nanoparticle,
wherein a second targeting moiety is attached to the capture moiety,
wherein the first targeting moiety has an affinity for a first portion of the target, wherein the second targeting moiety has an affinity for a second portion of the target, wherein the first portion of the target and the second portion of the target are not the same;
forming a probe complex including the probe, the second targeting moiety, and the target, wherein the first targeting moiety is attached to the first portion of the target, wherein the second targeting moiety is attached to the second portion of the target;
separating the probe complex from the other components of the mixture;
heating the probe complex; and
determining the melting point of the phase change material, wherein the melting point is used to identify the phase change material, wherein identifying the phase change material is used to determine the target.

31. The method of claim 30, wherein the capture moiety is a magnetic capture moiety, wherein the second targeting moiety is attached to the magnetic capture moiety, wherein the probe complex includes the second targeting moiety and the magnetic capture moiety.

32. The method of claim 30, wherein the capture moiety is a substrate, wherein the second targeting moiety is attached to the substrate.

33. The method of claim 32, wherein the substrate is a substrate selected from the group consisting of: an aluminum substrate, a gold substrate, a ceramic substrate, a glass substrate, a silicon dioxide substrate, and a silicon substrate.

34. A method of detecting a target, comprising:
mixing a first probe, a second probe, a first capture moiety, a first target, and a second target,
wherein the first probe is a nanoparticle made of a first phase change material, wherein a first targeting moiety is attached to the surface of the nanoparticle,
wherein the second probe is a nanoparticle made of a second phase change material, wherein the melting point of the first phase change material is different than the melting point of the second phase change material, wherein a second targeting moiety is attached to the surface of the nanoparticle,
wherein a third targeting moiety is attached to the first capture moiety,
wherein the first targeting moiety has an affinity for a first portion of the first target, wherein the third targeting moiety has an affinity for a second portion of the first target, wherein the first portion of the first target and the second portion of the first target are not the same,
wherein the second targeting moiety has an affinity for a first portion of the second target, wherein the third targeting moiety has an affinity for a second portion of the second target, wherein the first portion of the second target and the second portion of the second target are not the same;
forming a first probe complex including the first probe, the third targeting moiety, and the first target, wherein first targeting moiety is attached to the first portion of the first target, wherein the third targeting moiety is attached to the second portion of the first target;
forming a second probe complex including the second probe, the third targeting moiety, and the second target, wherein the second targeting moiety is attached to the first portion of the second target, wherein the third targeting moiety is attached to the second portion of the second target;
separating the first probe complex and the second probe complex from the other components of the mixture;
heating the first probe complex and the second probe complex;
determining a first melting point of the first phase change material, wherein the first melting point is used to identify the first phase change material, wherein identifying the first phase change material is used to determine the first target; and
determining a second melting point of the second phase change material, wherein the second melting point is used to identify the second phase change material, wherein identifying the second phase change material is used to determine the second target.

35. A method of detecting a target, comprising:
mixing a first probe, a second probe, a first capture moiety, a second capture moiety, a first target, and a second target,
wherein the first probe is a nanoparticle made of a first phase change material, wherein a first targeting moiety is attached to the surface of the nanoparticle,
wherein the second probe is a nanoparticle made of a second phase change material, wherein the melting point of the first phase change material is different than the melting point of the second phase change material, wherein a second targeting moiety is attached to the surface of the nanoparticle,
wherein a third targeting moiety is attached to the first capture moiety,
wherein a fourth targeting moiety is attached to the second capture moiety,
wherein the first targeting moiety has an affinity for a first portion of the first target, wherein the third targeting moiety has an affinity for a second portion of the first target, wherein the first portion of the first target and the second portion of the first target are not the same,
wherein the second targeting moiety has an affinity for a first portion of the second target, wherein the fourth targeting moiety has an affinity for a second portion of the second target, wherein the first portion of the second target and the second portion of the second target are not the same;
forming a first probe complex including the first probe, the third targeting moiety, and the first target, wherein first targeting moiety is attached to the first portion of the first target, wherein the third targeting moiety is attached to the second portion of the first target;
forming a second probe complex including the second probe, the fourth targeting moiety, and the second target, wherein the second targeting moiety is attached to the first portion of the second target, wherein the fourth targeting moiety is attached to the second portion of the second target;
separating the first probe complex and the second probe complex from the other components of the mixture;
heating the first probe complex and the second probe complex;
determining a first melting point of the first phase change material, wherein the first melting point is used to identify the first phase change material, wherein identifying the first phase change material is used to determine the first target; and
determining a second melting point of the second phase change material, wherein the second melting point is used to identify the second phase change material, wherein identifying the second phase change material is used to determine the second target.
